(19) United States
(12) Patent Application Publication
(10) Pub. No.: US 2026/0294713 A1
(43) Pub. Date: Oct. 1, 2026

(54) Individually Adjusted Inflatable Zone Support System

(71) Applicant: Xsensor Technology Corporation, Calgary (CA)

(72) Inventors: Ian Main, Calgary (CA); Bruce L. Malkinson, Calgary (CA); Dylan B. Heckbert, Calgary (CA); Shannon I. Blyth, Calgary (CA)

(21) Appl. No.: 19/632,148

(22) Filed: Mar. 27, 2026

Related U.S. Application Data
(60) Provisional application No. 63/779,996, filed on Mar. 28, 2025.

(51) Int. Cl. A61G 7/057; A61B 5/00

(52) U.S. Cl. CPC A61G 7/05776; A61B 5/447; A61B 5/7267; A61B 5/7275; A61G 2203/34

(57) ABSTRACT

In some embodiments, a weight support system may include a weight support device having a sensor grid configured to generate location-specific pressure data and a plurality of inflatable zones that may be individually adjustable by one or more air pumps under control of a computer. The computer may receive the location-specific pressure data, may determine that a portion of a subject's body corresponding to a first inflatable zone is at risk of a pressure injury, and may cause an adjustment of the first inflatable zone to reduce the risk. In some embodiments, at least a second inflatable zone maintains inflation during the adjustment of the first inflatable zone to maintain support of the subject.

### CROSS-REFERENCE TO RELATED APPLICATION(S)

This application claims the benefit of U.S. Provisional Application No. 63/779,996, filed Mar. 28, 2025, which is incorporated by reference in its entirety.

### TECHNICAL FIELD

The present disclosure generally relates to a weight support device, in particular to a weight support device that includes a sensor grid that can detect pressure data of a person.

### BACKGROUND

Pressure injuries (e.g., bedsores, pressure ulcers, pressure sores, or decubitus ulcers) are a common problem in clinical or at-home settings. Oftentimes, the presence of a pressure injury in a subject may lead to an increase in time spent at a hospital, an increase in medical costs (e.g., Medicare no longer covers the cost of hospital-acquired pressure ulcers), and/or permanent damage suffered by the subject. There are four stages of pressure injury: Stage I—The skin is a slightly different color, but there are no open wounds, Stage II—The skin breaks open and an ulcer forms, Stage III—The sore becomes worse and creates a crater in the tissue, and Stage IV—The sore is very deep causing extensive damage and can harm muscle, bone and tendons. Stage III and IV ulcers can lead to serious complications such as infections of the bone or blood (sepsis). Depending on the healthcare region, these injuries are considered ‘never events’, which are subject safety incidents that result in serious subject harm or death, and that can be prevented by using organizational checks and balances.

A typical prevention strategy to avoid pressure injury in a subject is a turn regimen where a healthcare professional or other caregiver re-positions the subject periodically to relieve localized pressure before the subject's skin integrity is breached and a wound begins to form. Unfortunately, currently-used turn regimens are not personalized to each subject or optimized for efficiency of the healthcare professional's time.

Additionally, injuries caused by a fall are common problems in clinical or at-home settings. For example, decreasing an amount of injuries caused by a fall from a wheelchair or from a bed is desirable in many subject settings. One subject setting where detecting a fall would provide an important service is in settings where high-risk populations like seniors are treated. Seniors and other high-risk populations are more prone to injury and extended periods of healing after a fall, thus detecting when a subject is about to fall and preventing the fall is important in reducing amount of time spent at a hospital for the subject, a decrease in medical costs, and/or prevention of any injury due to fall for those subjects.

### SUMMARY

In some embodiments, the disclosure described herein relate to a weight support system for a subject, the weight support system including: a weight support device configured to support the subject, the weight support device including: a sensor grid configured to generate location-specific pressure data; and a plurality of inflatable zones, wherein at least a subset of the inflatable zones are individually adjustable; one or more air pumps configured to provide airflow to the plurality of inflatable zones; and a computer configured to control adjustments of the plurality of inflatable zones, wherein controlling the adjustments of the plurality of inflatable zones includes: receiving the location-specific pressure data from the sensor grid; determining, using the location-specific pressure data, that a portion of the subject's body corresponding to a first inflatable zone is at risk of a pressure injury; and causing an adjustment of the first inflatable zone corresponding to the portion of the subject's body to reduce the risk of the pressure injury of the subject, wherein at least a second inflatable zone of the plurality of inflatable zones is maintained during the adjustment of the first inflatable zone.

In some embodiments, the disclosure described herein relates to a weight support system, further including a plurality of transducers, wherein each transducer is configured to monitor an adjustment of one of the plurality of inflatable zones.

In some embodiments, the disclosure described herein relates to a weight support system, further including a plurality of airflow valves, wherein each airflow valve is configured to control airflow to one of the plurality of inflatable zones.

In some embodiments, the disclosure described herein relates to a weight support system, wherein the plurality of inflatable zones includes: a first subset of longitudinally arranged inflatable zones configured to support a sacral region of the subject; a second subset of latitudinally arranged inflatable zones configured to support an upper body region of the subject; and a third subset of latitudinally arranged inflatable zones configured to support a lower body region of the subject.

In some embodiments, the disclosure described herein relates to a weight support system, wherein the computer is configured to control adjustments for the plurality of inflatable zones using a machine-learning model, wherein the machine-learning model is configured to receive the location-specific pressure data and outputs a prediction describing one or more locations on the sensor grid that exceed a pressure threshold and the portion of the subject's body corresponding to the one or more locations.

In some embodiments, the disclosure described herein relates to a weight support system, wherein the computer is configured to control adjustments for the plurality of inflatable zones using a machine-learning model, wherein the machine-learning model is trained to identify that a portion of the subject's body is at risk of a pressure injury based on the pressure data for one or more locations on the sensor grid.

In some embodiments, the disclosure described herein relates to a method for supporting a subject using a plurality of inflatable zones including: receiving location-specific pressure data from a sensor grid from a weight support device, the weight support device including: a sensor grid configured to generate the location-specific pressure data; and a plurality of inflatable zones, wherein at least a subset of the inflatable zones are individually adjustable by one or more air pumps configured to provide airflow to the plurality of inflatable zones; determining, using the location-specific pressure data, that a portion of the subject's body corresponding to a first inflatable zone is at risk of a pressure injury; and causing an adjustment of the first inflatable zone corresponding to the portion of the subject's body to reduce the risk of the pressure injury of the subject, wherein at least a second inflatable zone of the plurality of inflatable zones is maintained during the adjustment of the first inflatable zone.

In some embodiments, the disclosure described herein relates to a method, further including: monitoring adjustments to the plurality of inflatable zones using a plurality of transducers.

In some embodiments, the disclosure described herein relates to a method, wherein causing an adjustment of the first inflatable zone includes controlling airflow for adjustments to the plurality of inflatable zones using a plurality of airflow valves.

In some embodiments, the disclosure described herein relates to a method, wherein the subset of the inflatable zones is configured to support at least a sacral region, an upper body region, or a lower body region of the subject.

In some embodiments, the disclosure described herein relates to a method, wherein determining that a portion of the subject's body is at risk of a pressure injury includes using a machine-learning model, wherein the machine-learning model is configured to receive location-specific pressure data and outputs a prediction describing one or more locations on the sensor grid that exceed a pressure threshold and the portion of the subject's body corresponding to the one or more locations.

In some embodiments, the disclosure described herein relates to a method, wherein determining that a portion of the subject's body is at risk of a pressure injury includes using a machine-learning model, wherein the machine-learning model is trained to identify that a portion of the subject's body is at risk of a pressure injury based on the pressure data for one or more locations on the sensor grid.

In some embodiments, the disclosure described herein relates to a mattress for a subject, the mattress including: a sensor grid configured to generate location-specific pressure data; a plurality of inflatable zones, wherein at least a subset of the inflatable zones are individually adjustable; a plurality of inlets configured to be coupled to one or more air pumps configured to provide airflow to the plurality of inflatable zones; and a transceiver configured to be in communication with a computer, the computer configured to control adjustments of the plurality of inflatable zones, wherein controlling the adjustments of the plurality of inflatable zones includes: receiving the location-specific pressure data from the sensor grid; determining, using the location-specific pressure data, that a portion of the subject's body corresponding to a first inflatable zone is at risk of a pressure injury; and causing an adjustment of the first inflatable zone corresponding to the portion of the subject's body to reduce the risk of the pressure injury of the subject, wherein at least a second inflatable zone of the plurality of inflatable zones is maintained during the adjustment of the first inflatable zone.

In some embodiments, the disclosure described herein relates to a mattress, wherein the first inflatable zone and the second inflatable zone of the plurality of inflatable zones are arranged in an alternating configuration.

In some embodiments, the disclosure described herein relates to a mattress, wherein the plurality of inflatable zones includes: a first subset of longitudinally arranged inflatable zones configured to support a sacral region of the subject; a second subset of latitudinally arranged inflatable zones configured to support an upper body region of the subject; and a third subset of latitudinally arranged inflatable zones configured to support a lower body region of the subject.

In some embodiments, the disclosure described herein relates to a mattress, wherein only the inflatable zones of the first subset of longitudinally arranged inflatable zones configured to support the sacral region of the subject are individually adjustable.

In some embodiments, the disclosure described herein relates to a mattress, further including an adjustable base inflatable zone configured to support the plurality of inflatable zones and to adjust the vertical height of the mattress based on the inflation of the base inflatable zone.

In some embodiments, the disclosure described herein relates to a mattress, further including a plurality of adjustable base inflatable zones.

In some embodiments, the disclosure described herein relates to a mattress, further including an adjustable bolster inflatable zone configured to provide lateral support for the subject.

In some embodiments, the disclosure described herein relates to a mattress, wherein the mattress includes a plurality of adjustable bolster inflatable zones.

### BRIEF DESCRIPTION OF THE DRAWINGS

Figure (FIG.) 1A is a block diagram illustrating an example system environment, in accordance with some embodiments.

FIG. 1B is a block diagram illustrating a continuity of care example system environment for a subject, in accordance with some embodiments.

FIGS. 2A, 2B, and 2C are block diagrams illustrating cross-sectional views of various example configurations of weight support devices, in accordance with some embodiments.

FIG. 2D is a conceptual diagram that shows several example top views of various weight support devices, in accordance with some embodiments.

FIG. 3A is a view of an example weight support system including a weight support device, in accordance with some embodiments.

FIG. 3B is a top view of an example weight support device comprising subsets of inflatable zones, in accordance with some embodiments.

FIG. 3C is a section side view of an example weight support device comprising subsets of inflatable zones, in accordance with some embodiments.

FIG. 3D is a section side view the inflatable zones, in accordance with some embodiments.

FIG. 4 is a block diagram illustrating an example weight support system including a weight support device, in accordance with some embodiments.

FIG. 5 is a flowchart depicting an example process for supporting a subject using a plurality of inflatable zones, in accordance with some embodiments.

FIG. 6 is a block diagram illustrating an example algorithmic pipeline for determining an adjustment of a plurality of inflatable zones based on pressure data, in accordance with some embodiments.

FIG. 7 is a structure of an example neural network, according to some embodiments.

FIG. 8 is a flowchart depicting an example region-specific offload process, in accordance with some embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

Embodiments described herein relate to a weight support device that functions as an intelligent surface (e.g., mattress, cushion, or seat) integrating pressure and moisture sensor grids, microclimate layers, and auxiliary sensors (accelerometers, thermistors, piezoelectric elements). Connected to local and cloud computing, the system maps contact pressure, identifies body outline and joint locations, detects high-shear and high-moisture areas, and predicts pressure-injury and fall outcomes using rules and machine learning.

The device may include an automatically adjustable surface that include multiple independently controlled inflatable zones, supplied by one or more air pumps and managed with airflow valves and transducers for precise pressure control. Using location-specific pressure data and ML-based classification, the system targets elevated pressure events with interventions such as sacral offloading (deflating a selected longitudinal zone group while adjacent groups remain inflated), heel flotation (selective deflation/inflation in lower-body latitudinal zones), and smart alternating pressure cycles to reduce local pressure toward near-zero while maintaining overall support. Adjustable base zones can lower bed height for ingress/egress, and bolster zones provide lateral support. Quick-connect inlets and wired/wireless connectivity enable flexible deployment and control.

Targeted sacral offloading uses the mattress's sensor grid to continuously map location-specific pressures and identify elevated pressure events in the sacral region. When a hotspot is detected, the computer-controlled system selectively deflates the group of longitudinal inflatable zones directly under the hotspot while keeping adjacent longitudinal groups inflated, shifting load away from the sacrum and driving local pressure toward near-zero (for example, <5 mmHg) for a defined interval before returning to baseline settings. This targeted, time-bounded offload can be triggered by thresholds or scheduled intervals and is designed to reduce sacral pressure-injury risk while maintaining overall support and stability for the subject.

Targeted heel floatation applies the same sensor-driven, zonal control to the lower body. If the sensor grid identifies elevated pressure at one or both heels, the system deflates the corresponding lower-body latitudinal zone group beneath the heels and maintains (or increases) inflation in the adjacent lower-body group to preserve limb support. Similar to sacral offloading, heel floatation reduces local pressure toward approximately zero for a set duration and then returns to preset pressures, minimizing heel pressure-injury risk without compromising comfort or posture. Detection can be rule-based or machine-learning-assisted, ensuring timely, location-specific intervention.

Smart alternating pressure zones proactively redistribute load across the upper and lower body to prevent prolonged high pressure. The system cycles groups of latitudinal zones in the upper and lower body, inflating adjacent groups to offload the highest-pressure locations without necessarily deflating the zone directly beneath those locations, and alternates support over set intervals. These cycles run independently of sacral offloading and heel floatation, enabling concurrent, region-specific strategies that adapt to changing pressure maps while preserving continuous support and subject stability.

### Example System Environments

Referring now to Figure (FIG.) 1A, shown is a block diagram illustrating an embodiment of an example system environment 100, in accordance with some embodiments. The components in the system environment 100 may be combined in the system with secure, password authenticated network transport, storage and retrieval of subject information compliant with healthcare information regulations and legislation. By way of example, the system environment 100 includes a weight support device 110, a hand-held sensor 115, an airflow system 120, a local computer 130, a computing server 140, a data store 155, a user device 160, a management device 170, and network 180. In various embodiments, the system environment 100 may include fewer or additional components. The system environment 100 also may include different components. Also, while some of the components in the system environment 100 may sometimes be described in a singular form, the system environment 100 may include one or more of each of the components.

The weight support device 110 may include layers that support the weight or part of the weight of a person (e.g., a subject) and include sensors that monitor various data associated with the person. Examples of the weight support device 110 include a bedding system (e.g., a mattress), a seating system (e.g., a wheelchair, a dining chair, an office chair, a car seat), a sheet, a cushion, a pillow, a pad, etc. The weight support device 110 may also be referred to as an intelligent surface. While in this disclosure the weight support device 110 is often described as a bedding system, various features and components of the bedding system may also be applied to other types of the weight support device 110 without explicitly referring to those types of the weight support device 110. The weight support device 110 may come in various sizes and different forms. For example, a bedding system may be an intelligent mattress that may include various comfort layers such as foam. In another example, a bedding system may be a pad that is intended to complement a conventional mattress (e.g., being laid on top of the mattress). A bedding system may also be used in a special setting such as in the hospital or elderly care facility. The weight support device 110 may also be a seating system that can be used as an intelligent office seat that monitors the posture of a person, a car seat (or a cushion for a car seat), or a wheelchair seat. Other examples of the weight support device 110 are also possible.

The weight support device 110 may include one or more types of sensors that are used to monitor various data about and certain vital information of the person sleeping, seating, or otherwise resting on the weight support device 110. The sensors may include a pressure sensor grid that includes an array of pressure sensing elements and a surface moisture sensor grid that includes an array of surface moisture sensing elements. The pressure sensing elements may take the form of resistive pressure sensors, fiber-optic pressure sensors, or capacitive pressure sensors. The surface moisture sensing elements may take the form of capacitive surface moisture sensors. The weight support device 110 may also include other types of sensors such as piezoelectric sensors, accelerometers, and thermistors.

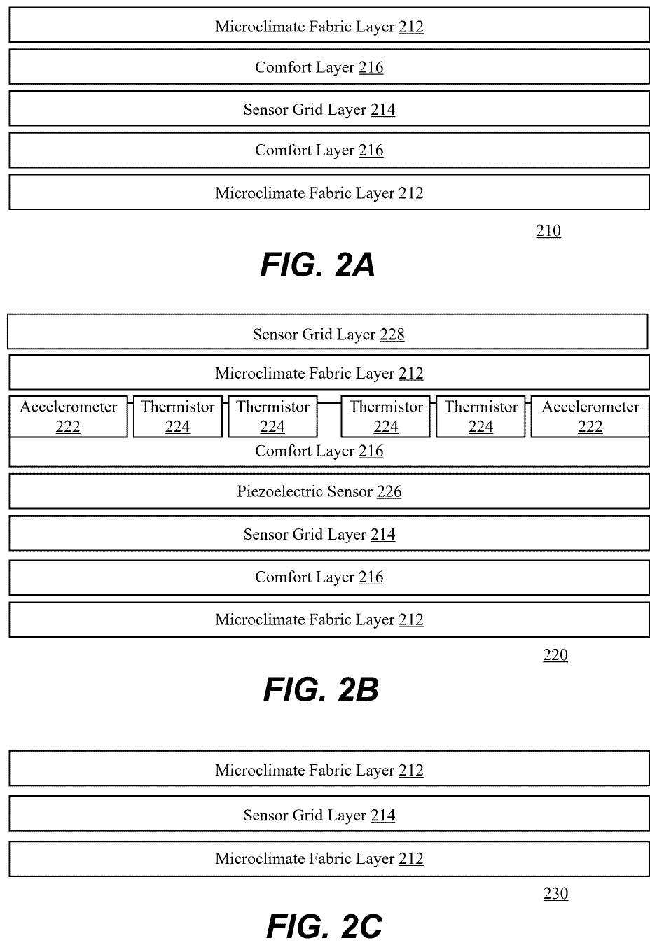

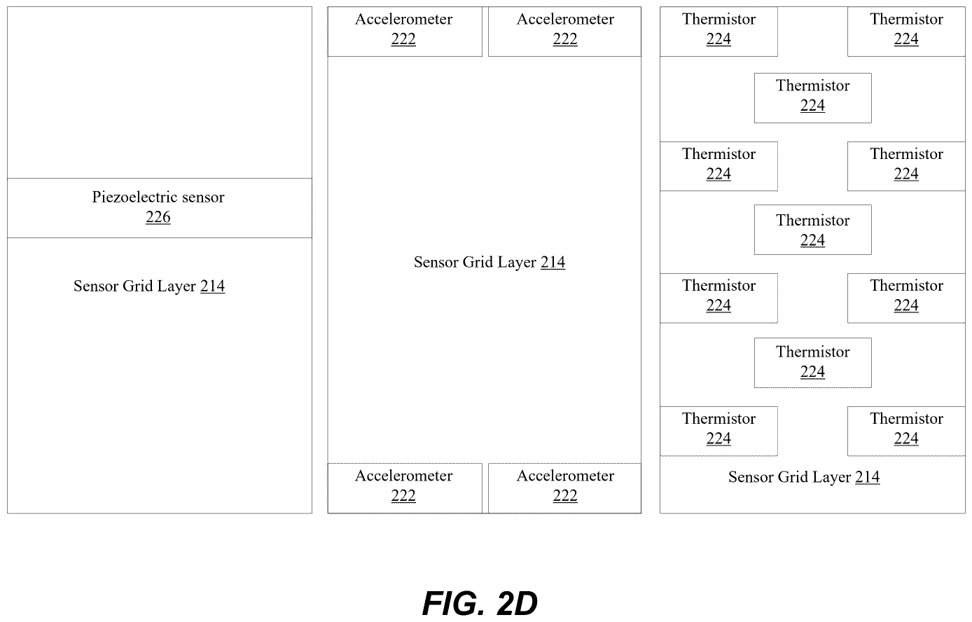

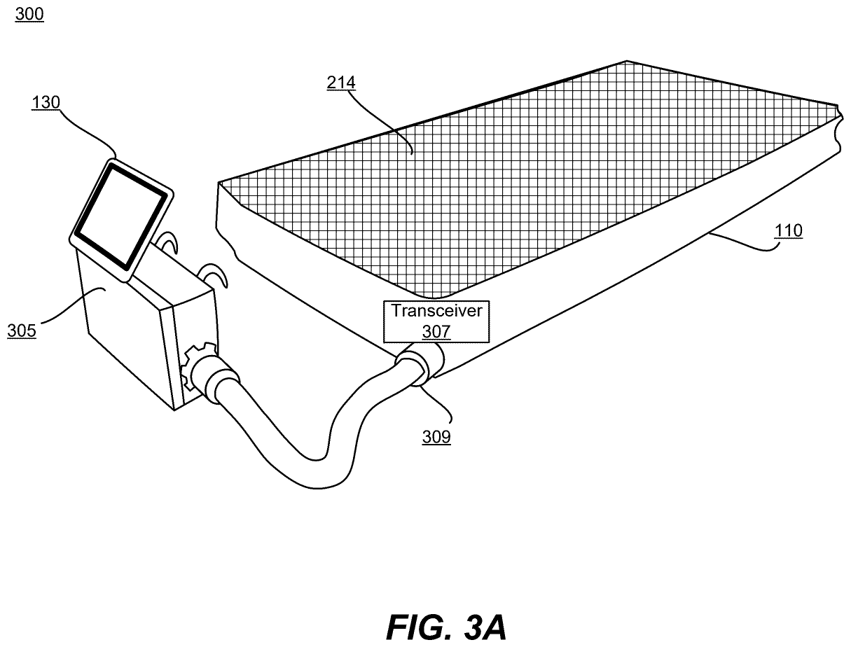

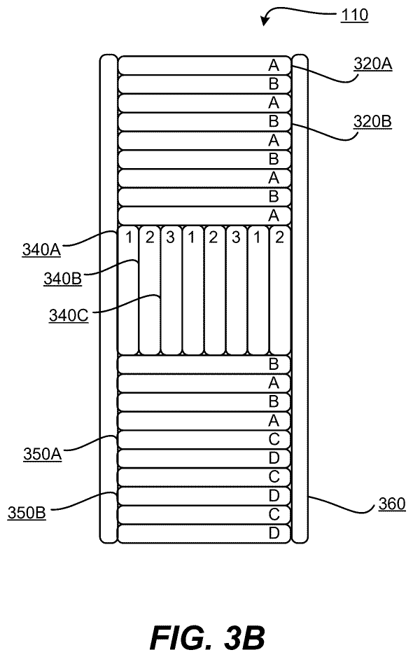

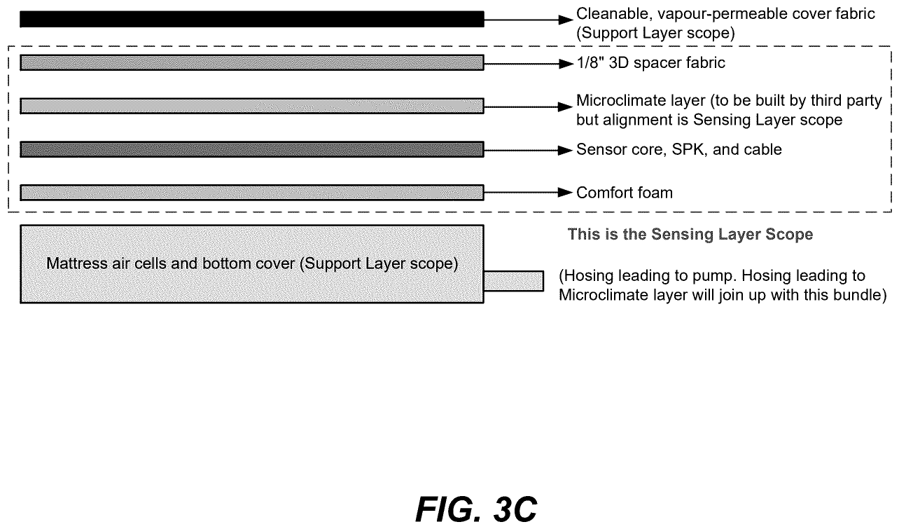

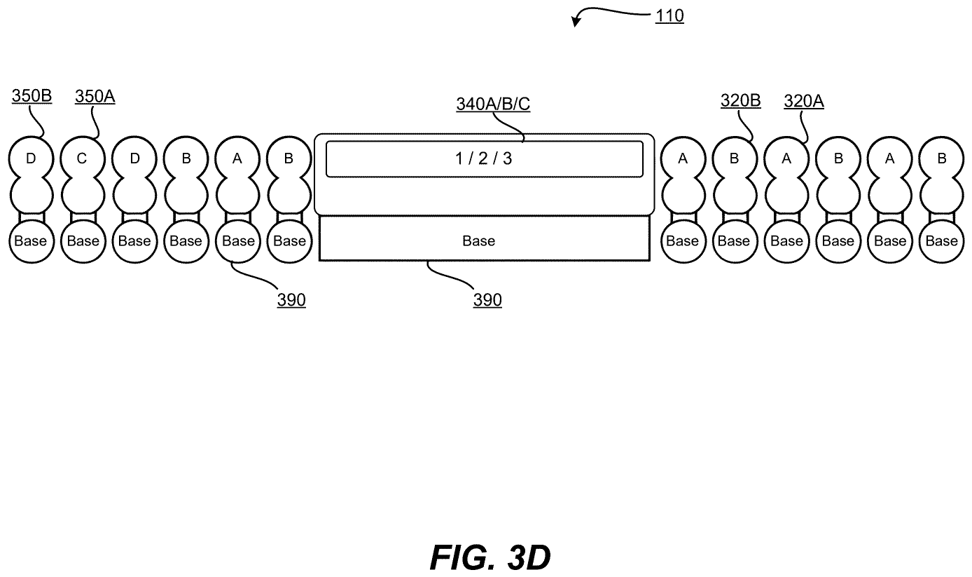

The weight support device 110 may include various comfort features such as one or more microclimate fabric layers that may be used to regulate the humidity, airflow, and temperature of the surface of the weight support device 110. The sensor grid(s) may also be formed from air permeable materials so that the sensor grid layer(s) is also air permeable. The weight support device 110 may also include a comfort layer that adjusts the firmness of the device. Structures and various components of different embodiments of the weight support device 110 will be discussed in further detail with reference to FIG. 2A through FIG. 3B.

The weight support device 110 may further include an inlet that can be connected to an active air source such as the airflow system 120 to enhance the air circulation and flow inside the weight support device 110. For example, the airflow system 120 may be a fan or a pump that moves air into some of the inner layers of the weight support device 110. In turn, the air is circulated through the air-permeable layers to exit the weight support device 110 through its surfaces. The airflow system 120 may be computer-controlled based on readings from temperature sensors and humidity sensors installed at the weight support device 110 to actively regulate the microclimate of the system. The weight support device 110 may also include various accessory devices such as temperature control devices, temperature sensors, white noise generators, audio sensors, biofeedback sensors, lighting controls, and light sensors. Communication and control of the accessory devices can be performed via a Universal Serial Bus (USB) port, Firewire port, or via Bluetooth or WiFi wireless connections. Some example additional comfort features are discussed in U.S. Patent Application Publication 2018/0027988, dated Feb. 1, 2018, entitled “Bedding System with a CNN Based Machine Vision Process,” which is incorporated by reference herein for all purposes.

The weight support device 110 may include an automatically adjustable surface. The weight support device 110 may receive instructions from the local computer 130, the computing server 140, or the management device 170 to adjust the surface such that a positioning of the person supported by the weight support device 110 is adjusted. For example, the surface may be adjusted via inflation bladders or servo motors. The weight support device 110 may automatically adjust its positioning, for example, by decreasing its height off of the ground. The weight support device 110 may additionally include one or more guard rails positioned on one or more sides of the weight support device 110. The guard rails may be automatically adjusted by the weight support device 110.

The weight support device 110 may generate sensor signals and be in communication with a computer to automatically detect a pressure injury outcome and/or a fall outcome of the person. The weight support device 110 may take the form of a portable flexible mat that can provide biometric information without any direct wiring connected to the person. The weight support device 110 may measure the pressure exerted by the person using a sensor grid to generate a matrix of pressure readings. The matrix of pressure readings and other sensor readings (e.g., surface moisture readings), which may be supplemented with other supporting sensors, may be provided to a computer with an artificial intelligence system which uses one or more types of machine learning networks (a convolutional neural network (CNN), a long short term memory (LSTM) network, etc.) to identify the pressure injury outcome (e.g., a risk of the person developing a pressure injury). The matrix of pressure readings and other sensor readings may also be used to deduce other information about the person that may include, but is not limited to, respiration rate, heart rate, and/or position data that includes body position, joint locations, and movement monitoring.

The person on the weight support device 110 may also be monitored by one or more hand-held sensors 115 that measure an amount of moisture under the person's skin and/or the person's vitals such as heart rate, respiration rate, body temperature, blood pressure, blood sugar level, etc. Depending on embodiments, the hand-held sensors 115 may be part of the weight support device 110 or independent devices. For example, in one embodiment, the weight support device 110 is equipped with sensors that can detect the heart rate, respiration rate, and body temperature. In another embodiment, the amount of moisture under the person's skin may be detected through an external hand-held sensor 115 that is not part of the weight support device 110. For example, the hand-held sensor 115 may be an edema sensor that takes the form of a device that transmits a radio frequency, an infrared frequency, or some other type of frequency light signal towards a portion of skin of the person and receive a reflected signal that provides information about the amount of moisture under the person's skin. For example, the hand-held sensor 115 may be an infrared tissue oxygenation sensor or a blood flow sensor. The hand-held sensors 115 may be professionally graded or customer graded. For example, in a hospital setting, the weight support device 110 may be used as a hospital bed for a subject who is monitored by different kinds of medically graded hand-held sensors 115. In some embodiments, the hand-held sensor 115 can inform a user (e.g., a healthcare professional) of a pressure injury outcome. In another example, one of the hand-held sensors 115 may simply be a wearable electronic device such as APPLE WATCH or a FITBIT smartwatch. For example, the heart rate of the person may be input from an external device that is embedded into the weight support device 110 or attached to the person outside of the weight support device 110. The hand-held sensors 115 may also include a pulse oximeter.

In some embodiments, the weight support device 110 and the hand-held sensors 115 may be connected to a local computer 130 that is located, for example, in the same place as the weight support device 110 (e.g., in a subject's hospital room). In some embodiments, the weight support device 110 may be equipped with processing power such as having built-in CPUs or the local computer 130 being part of the weight support device 110. In other embodiments, the local computer 130 may be a separate computer that connects to the weight support device 110 and the hand-held sensors 115 to collect data from those devices. The local computer 130 may upload the data via the network 180 to the computing server 140 for further processing. In some embodiments, the local computer 130 may also have software installed to analyze the data from the weight support device 110 and/or the hand-held sensors 115. For example, in a hospital setting, the local computer 130 may be a bedside monitor that provides analyses of various data in real-time and display the data associated the subject. In other embodiments, the local computer 130 simply collect data or perform certain data processing (such as compression, conversion of formats) for the computing server 140 to further analyze the data. The role of the local computer 130 may vary in different implementations and settings. In some embodiments, local computer 130 may not be present. For example, the weight support device 110 may be equipped with a wireless capability that can directly transmit its data to the computing server 140 for processing.

In some embodiments, a weight support device 110 (or a computer that processes the raw data of the weight support device 110) may transmit data such as visual representations of motions and positions/poses of the person, pressure data, surface moisture data, and/or surface temperature data in a secure network environment to a user (e.g., a healthcare professional) via a management dashboard (e.g., at a nursing station, front management desk in a retirement home, etc.) of a management device 170 to highlight the state and status of the individual being monitored. The management device 170 may also provide an alert system alerting the user that a subject is at risk of developing a pressure injury and/or at risk of falling off of the weight support device 110, that an adjustment of a positioning of the subject is needed, when the adjustment is needed, etc. The management device 170 may be communicatively coupled to more than one weight support device 110 and provide the status of multiple individuals to the user. The management device 170 may prioritize the individuals and provide their status accordingly based on each individual's risk of developing a pressure injury and/or risk of falling, when each individual needs their positioning adjusted, and/or how long an area or areas of each individual has been experiencing high-pressure. Also, the alert system may alert the user should there be any trends or behavior outside of preestablished parameters (e.g., oxygen level).

The computing server 140 may be a remote server that is used to analyze data collected from the weight support device 110 and the hand-held sensors 115 to predict a pressure injury outcome and/or a fall outcome of a person supported by the weight support device 110. The computing server 140 may take the form of a combination of hardware and software, such as engines 142 through 152. The computing server 140 may take different forms. In one embodiment, the computing server 140 may be a server computer that executes code instructions to cause one or more processors to perform various processes described herein. In another case, the computing server 140 may be a pool of computing devices that may be located at the same geographical location (e.g., in a server room) or be distributed geographically (e.g., cloud computing, distributed computing, or in a virtual server network). The computing server 140 may also include one or more virtualization instances such as a container, a virtual machine, a virtual private server, a virtual kernel, or another suitable virtualization instance.

The computing server 140 may include various algorithms for data analysis. The sensor mapping engine 142 may perform basic functions such as receiving data from the weight support device 110 and the hand-held sensors 115 and organizing the received data. For example, the sensor mapping engine 142 may organize the data measured by the array of pressure sensing elements into an array of measurements representative of the sensor array. The sensor mapping engine 140 may generate a visual representation of the array of sensor measurements.

The sensor mapping engine 142 may also calculate a number of parameters that are derived from the image representation. For example, a contact area can be calculated for the entire sensing area of the weight support device 110. The contact area is based on areas that correspond to pressure measurements above a minimum threshold. The contact area may provide a visual outline (e.g., a body outline) of the individual supported by the weight support device 110. Based on the body outline of the individual, the sensor mapping engine 142 may provide instruction to the weight support device 110 to only collect sensor data from the surface moisture sensing elements and/or the thermistors located on the weight support device 110 within the contact area.

In another example, the sensor mapping engine 142 may calculate an average peak pressure over the entire sensing area of the weight support device 110. In one approach, the sensor mapping engine 142 may calculate an average peak pressure by isolating a group of sensing points with the highest measured pressures (the peak pressures), then averaging those pressure values to obtain the result. A sensing point is an individual sensing element within the sensor array. For example, using a bed sensor with 1664 sensing points in the sensor area, the 16 sensing points with the highest pressure measurements could be averaged to determine the average peak pressure. The number of sensing points averaged could be 25% to 0.5%, or preferably 1%, of the total number of sensing points in the array. The number of sensing points averaged could also be 25% to 0.5%, or preferably 1%, of the total number of sensing points in the array that are above a pressure threshold, for example, 10 mmHg. The sensor mapping engine 142 may reject certain peak pressures in order to reduce the impact of creases in the sensor grid, objects in the customer's pockets, or hard edges in the customer's clothing. For example, the three highest pressure measurements can be excluded from the average peak pressure calculation.

The sensor mapping engine 142 may calculate a load calculation (e.g., another pressure-related parameter) based on the sensor data. For example, a load calculation could be used to estimate a person's weight. The sensor mapping engine 142 may estimate the person's height by adding the number of sensing points associated with a minimum pressure from the person's head to their toes when they are lying on their back.

The sensor mapping engine 142 may determine one or more areas of high shear (e.g., another pressure-related parameter). In some embodiments, the sensor mapping engine 142 determines an area of high shear based on pressure gradients between sensing points. For example, a gradient between adjacent sensing points that is greater than a threshold gradient value corresponds to an area with high shear. In some embodiments, the sensor mapping engine 142 determines one or more areas of high shear based on surface contour of the weight support device 110.

The sensor mapping engine 142 may generate a visual representation of the array of sensor measurements related to surface moisture. The sensor mapping engine 142 may overlay the visual representation of the surface of the weight support device 110 with the visual representation of the person. The sensor mapping engine 142 may detect areas where the outline of the person overlaps with areas of high-moisture.

The machine vision engine 144 analyzes the arrays of sensor measurements, visual representations, and other parameters determined by the sensor mapping engine 142 to identify body types and to identify body position of the person supported by the weight support device 110. The machine vision engine 144 may include one or more algorithms (which could include AI or not) that extracts features from the information received from the sensor mapping engine 142. For example, when a person first lies on the weight support device 110 (as a bedding system), the machine vision engine 144 analyzes the two-dimensional visual representation of the person and derives a physical profile. The machine vision engine 144 may match the physical profile to a physical profile previously stored in the data store 155. The machine vision engine 144 may determine an identity of the person and pass this information to other components. The weight support device 110 can then be configured appropriately for that person based on information in their physical profile.

A “physical profile” is at least one physical attribute of an individual and may include attributes such as measurements of certain body features, for example, height, weight, shoulder-width, hip-width or waist-width; or ratios of these measurements, for example, shoulder to hip ratio, shoulder to waist ratio, or waist to hip ratio; body type, for example, endomorph, ectomorph, endomorph; or Body Mass Index (BMI).

The machine vision engine 144 may calculate a mass distribution for the person. For example, the machine vision engine 144 creates a peak pressure curve along a length of a person lying on their back or side. The mass distribution may be calculated from applied pressure over a given unit area. For example, the machine vision engine 144 may calculate a mass for each individual sensing point in the sensing array by multiplying the measured pressure by the area of the sensing point. Mass can also be calculated for larger areas by averaging pressure measurements over a group of sensing points, for example, 2×2 or 4×4 sensing points. The machine vision engine 144 can create a body mass curve along the length of a person lying on their back or side. The peak pressure curve and/or the body mass curve can also be used for matching a person to their physical profile. The machine vision engine 144 may calculate a center of mass for the person based on the mass calculations for all areas within the contact area and a position of each area in the contact area.

The machine learning engine 146 can detect positions and poses of the person and target body parts of the person supported by the weight support device 110. The machine learning engine 146 can continuously monitor and process the pressure data to determine a person's body position and pose. For example, position classifications can include “on back,” “left side,” or “right side.” The machine learning engine 146 may also determine the joint locations of the person and the movement of the person. For example, changes in pressure may indicate movement or restlessness. For example, if the pressure sensing points in the contact area above a minimum pressure threshold show little variation over a period of time, then the person can be considered motionless. A variation threshold of 10% to 100% of the measured pressure can be used to determine if there is movement on a particular sensing point or group of sensing points.

The vital analysis engine 148 analyzes data from the weight support device 110 and the hand-held sensors 115 to determine one or more biometrics that describe the vitals of the person. For example, the vital analysis engine 148 may rely on the machine learning engine 146 and the machine vision engine 144 to determine a target body part of the person. The vital analysis engine 148 may focus on sensor data associated with the target body part to determine certain vital information of the person.

The pressure injury outcome engine 150 may be part of the machine learning engine 146 and may use information from the sensor mapping engine 142, the machine vision engine 144, the machine learning engine 146, and the vital analysis engine 148 to predict a pressure injury outcome for a person supported by the weight support device 110. The pressure injury outcome can include a risk of the person developing a pressure injury, an area of the person at risk of developing the pressure injury, and/or an amount of time that indicates when an adjustment of the positioning of the person is needed to help avoid pressure injury.

In some embodiments, the pressure injury outcome engine 150 may utilize a rules-based (or heuristics-based approach) to predict the pressure injury outcome. An example rules-based approach for predicting pressure injury outcome is discussed in U.S. Pat. No. 9,320,665, patented on Apr. 26, 2016, entitled “Risk Modeling for Pressure Ulcer Formation,” which is incorporated by reference herein for all purposes.

In some embodiments, the pressure injury outcome engine 150 may utilize a machine learning model to predict the pressure injury outcome. Inputs into the machine learning model may include at least the raw sensor data measured by the pressure sensing elements. The inputs may further include one or more of: raw sensor data measured by the surface moisture sensing elements (e.g., the moisture sensing elements included in the weight support device 110), raw surface temperature data generated from the thermistors, a health record of an individual, sensor data from the hand-held sensors 115 (e.g., moisture data from a hand-held edema sensor), any additional information input by a user (e.g., results of a blanch test), information from the sensor mapping engine 142, and information from the machine visions engine 146.

In some embodiments, the pressure injury outcome engine 150 provides an alert, notification, and/or message to a user (e.g., a nurse or a caregiver) that the positioning of the person should be adjusted. In some examples, the notification may instruct the user at what time the adjustment should take place and/or which area(s) of the person needs its position adjusted. The notification may also provide a visual representation to the user of the person with at least one area of the person's body called out as the area of the person that needs positioning adjustment.

In some embodiments, the pressure injury outcome engine 150 provides instructions to the weight support device 110 based on the pressure injury outcome determination. For example, the pressure injury outcome engine 150 may instruct the weight support device 110 to adjust the positioning of the person.

The fall outcome engine 152 may be part of the machine learning engine 146 and may use information from the sensor mapping engine 142, the machine vision engine 144, and the machine learning engine 146 to predict a fall outcome for a person supported by the weight support device 110. The fall outcome can include a risk of the person falling off of the weight support device 110 and/or an indication that the person has experienced a fall.

In some embodiments, the fall outcome engine 152 may utilize a rule-based approach for predicting the fall outcome. In some embodiments, the fall outcome engine 152 may utilize a machine learning model to predict the fall outcome. Inputs into the rule-based approach or the machine learning model may include at least the raw sensor data measured by the pressure sensing elements (e.g., pressure readings). The inputs may further include one or more of: a health record of an individual, information from the sensor mapping engine 142, and information from the machine visions engine 146. In some embodiments, the fall outcome engine 152 provides an alert, notification, and/or message to the user that the positioning of the person should be adjusted to avoid a fall, or the person needs assistance after experiencing a fall. In some examples, the notification may instruct the user how best to adjust the positioning of the person.

In some embodiments, the fall outcome engine 152 provides instructions to the weight support device 110 based on the fall outcome determination. For example, the fall outcome engine 152 may instruct the weight support device 110 to adjust the positioning of the person.

The data store 155 includes one or more storage units such as memory that takes the form of non-transitory and non-volatile computer storage medium to store various data that may be uploaded by the local computer 130, by the weight support device 110, or by other components of the system environment 100. The computer-readable storage medium is a medium that does not include a transitory medium such as a propagating signal or a carrier wave.

The data store 155 may store health records of person(s) supported by the weight support device 110. The health records may have been input into the data store 155 by the local computer 130, the user device 160, the management device 170, etc. at any time. Each health record corresponds to a particular person and includes information about the person, such as an age, mobility information, nutrition information, pre-existing skin conditions, incontinent issues, medical history, current medications, results of blanch test, etc. The health record may also include information about one or more areas of the person (e.g., a wound site, a surgical site, etc.) that are to avoid pressure. The data store 155 may store sensor data (e.g., pressure data) captured by the weight support device 110 and also analysis results generated by the computing server 140, such as determined position data, pressure injury outcome(s), and/or fall outcome(s). The sensor data and analysis results corresponding to a particular person may be associated with a health record of that person and stored within the health record. In some embodiments, the data store 155 aggregates sensor data received from multiple weight support devices 110 by which the person has been supported. For example, in a hospital setting, a subject may be admitted to the hospital by a wheelchair, be treated on a first bedding system (e.g., a stretcher) in an emergency care and be transferred to a second bedding system (e.g., a hospital bed) in a subject room. The data store 155 may receive data from the wheelchair, the first bedding system, and the second bedding system for the computing server 140 to continuously monitor the pressure readings related to the subject. The data store 155 may additionally store sensor data received from the hand-held sensors 115.

The data store 155 may store historical subject data. The historical subject data includes health record data, sensors data, and analysis results for subjects that have historically been supported by the weight support device 110. The historical subject data may be utilized by one or more machine learning models to train the models to determine pressure injury outcomes and/or fall outcomes for current or future subjects.

The data store 155 may take various forms. In one embodiment, the data store 155 communicates with other components by the network 180. This type of data store 155 may be referred to as a cloud storage server. Example cloud storage service providers may include AWS, AZURE STORAGE, GOOGLE CLOUD STORAGE, etc. In another embodiment, instead of a cloud storage server, the data store 155 is a storage device that is controlled and connected to the computing server 140. For example, the data store 155 may take the form of memory (e.g., hard drives, flash memories, discs, ROMs, etc.) used by the computing server 140 such as storage devices in a storage server room that is operated by the computing server 140.

The user device 160 may be a portable electronic device for transmitting data. The user device 160 may be possessed by the person using (e.g., supported by) the weight support device 110. The user device 160 may be possessed by a different user in the system environment 100. For example, the user device 160 may be used by a healthcare professional, caregiver, etc. Examples of user devices 160 include personal computers (PCs), desktop computers, laptop computers, tablet computers, smartphones, wearable electronic devices such as smartwatches, or any other suitable electronic devices. The user device 160 may include an application such as a software application provided by the computing server 140. The application may provide various results and analyses of the sensor data collected by the weight support device 110 and hand-held sensor 115 and may also allow the person and/or the user to adjust various settings associated with weight support device 110, such as the airflow system 120. An application may be of different types. In one case, an application may be a web application that runs on JavaScript, etc. In the case of a web application, the application cooperates with a web browser to render a front-end interface 165. In another case, an application may be a mobile application. For example, the mobile application may run on Swift for iOS and other APPLE operating systems or on JAVA or another suitable language for ANDROID systems. In yet another case, an application may be a software program that operates on a desktop computer that runs on an operating system such as LINUX, MICROSOFT WINDOWS, MAC OS, or CHROME OS.

An interface 165 may be a suitable interface for the local computer 130 and/or the user device 160 to interact with the computing server 140. The interface 165 may include various visualizations and graphical elements to display notifications and/or information to users and may also include input fields to accept inputs from users. A user may communicate to the application and the computing server 140 through the interface 165. The interface 165 may take different forms. In one embodiment, the interface 165 may be a web browser such as CHROME, FIREFOX, SAFARI, INTERNET EXPLORER, EDGE, etc. and the application may be a web application that is run by the web browser. In another application, the interface 165 is part of the application. For example, the interface 165 may be the front-end component of a mobile application or a desktop application. The interface 165 also may be referred to as a graphical user interface (GUI) which includes graphical elements to display a digital heatmap, other pressure injury-related information, or other fall-related information. In another embodiment, the interface 165 may not include graphical elements but may communicate with the computing server 140 via other suitable ways such as application program interfaces (APIs).

The various functionalities of the computing server 140 may also be performed by the local computer 130 or the user device 160, depending on the implementation and configuration. For example, the software algorithms that perform the various process associated with engines 142, 144, 146, 148, 150, and 152 in the computing server 140 may also reside in the local computer 130 or a mobile application of the user device 160 so that the local computer 130 or the user device 160 may directly analyze the sensor data generated by the weight support device 110. Results generated may be displayed at the user device 160, at the local computer 130, or at both devices. The computing server 140 may manage a mobile application that can cause the local computer 130 or the user device 160 to generate a user interface 165 that displays various results, predictions, determinations, notifications, visual representations, and graphical illustrations of sensor data generated by the weight support device 110. In some embodiments, the weight support device 110 may also include computing components and software for analyzing the data directly and display the results.

The network 180 provides connections to the components of the system environment 100 through one or more sub-networks, which may include any combination of the local area and/or wide area networks, using both wired and/or wireless communication systems. In one embodiment, the network 180 use standard communications technologies and/or protocols. For example, a network 180 may include communication links using technologies such as Ethernet, 802.11, worldwide interoperability for microwave access (WiMAX), 3G, 4G, Long Term Evolution (LTE), 5G, code division multiple access (CDMA), digital subscriber line (DSL), etc. Examples of network protocols used for communicating via the network 180 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over a network 180 may be represented using any suitable format, such as hypertext markup language (HTML), extensible markup language (XML), JavaScript object notation (JSON), structured query language (SQL). In some embodiments, all or some of the communication links of a network 180 may be encrypted using any suitable technique or techniques such as secure sockets layer (SSL), transport layer security (TLS), virtual private networks (VPNs), Internet Protocol security (IPsec), etc. The network 180 also include links and packet switching networks such as the Internet.

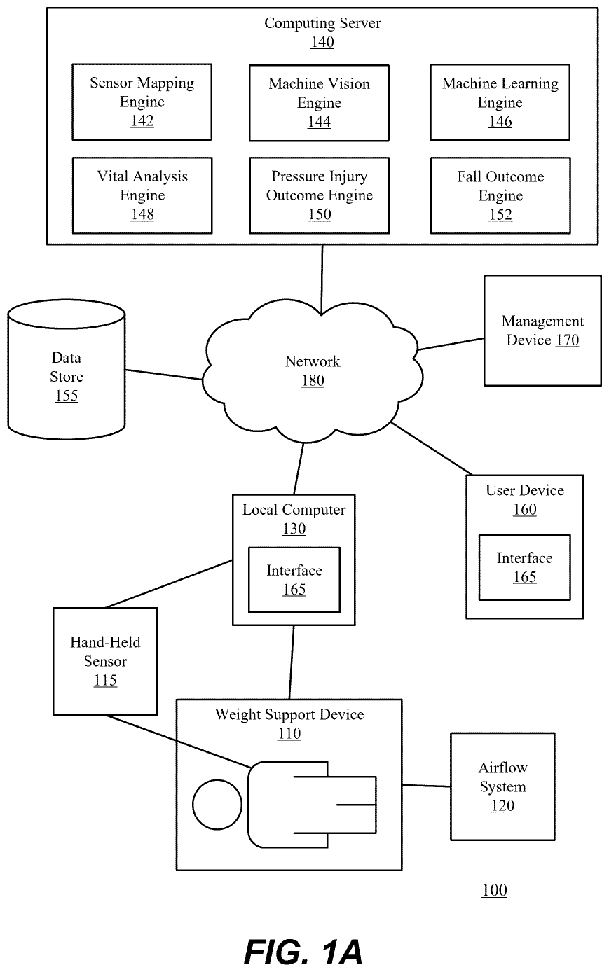

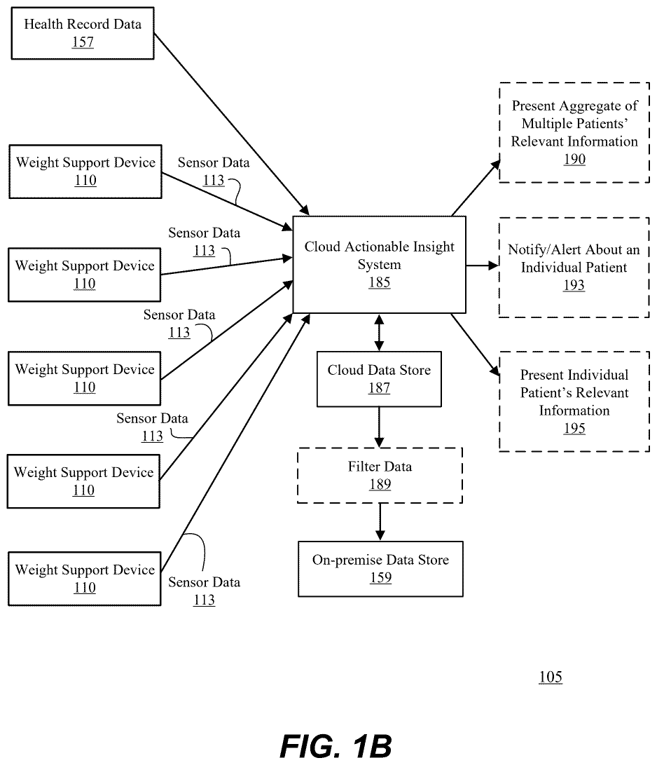

FIG. 1B is a block diagram illustrating a continuity of care example system environment 105 for a subject, in accordance with some embodiments. In some example scenarios of subjects'stays at a hospital, the subjects coming out of an operating room may eventually develop a pressure injury while in a hospital bed recovering from surgery-but the injury may have actually started with the subject experiencing high pressures while sedated in the operating room during their surgery. In a hospital setting with a multitude of subjects, each subject can be supported by different weight support devices 110 throughout their stay (e.g., from a wheelchair, to a first bedding system, to a second bedding system, and so on), a system to monitor and track the pressure injury outcomes may provide better visibility regarding where a pressure injury or risk of pressure injury first started. The system to monitor and track the fall outcomes may provide better visibility regarding how best to position a subject to avoid a fall from the weight support devices 110.

The continuity of care example system environment 105 for a subject tracks sensor data 113 received from various weight support devices 110. The sensor data 113 may include pressure data, surface moisture data, surface temperature data, and any other data measured by each weight support device 110. Additional data may be supplied by the data store 155. For example, the health record data 157 associated with the subject is monitored in the system environment 105. The sensor data 113 and health record data 163 are provided to a cloud actionable insight system 185 that may include the computing server 140. The cloud actionable insight system 185 may store the sensor data 113 and the health record data 157 in the cloud data store 187. The data in the cloud data store 187 may be filtered 189 and stored in the on-premise data store 159 (e.g., at the hospital). The cloud actionable insight system 185 analyzes the sensor data 113 and the health record data 157 to determine a pressure injury outcome and/or a fall outcome for the subject as described above.

In some embodiments, the cloud actionable insight system 185 presents 190 an aggregate of the multiple subjects'relevant information to the healthcare professional and/or caregivers via the local computer 130, the user device 160, and/or the management device 170. In some embodiments, the cloud actionable insight system 185 may prioritize how and/or when each subject's relevant information is presented. For example, the cloud actionable insight system 185 may determine to display information about a subject with a higher risk of developing a pressure injury and/or a subject at risk of developing a pressure injury sooner to the healthcare professional before displaying information about a subject with a lower risk and/or a subject at risk of developing a pressure injury later. In another example, the cloud actionable insight system 185 may determine to display information about a subject with a higher risk of experiencing a fall and/or a subject at risk of experiencing a fall sooner to the healthcare professional before displaying information about a subject with a lower risk and/or a subject at risk of experiencing a fall later. The relevant information may include a visual representation of all subjects currently being support by a weight support device 110 and any corresponding pressure injury outcomes and/or fall outcomes for those subjects.

In some embodiments, the cloud actionable insight system 185 may notify and/or alert 193 the healthcare professional, caregivers, and/or subject via the local computer 130, the user device 160, and/or the management device 170 that a subject is at risk of developing a pressure injury and/or at risk of falling off of the weight support device 110. The notification and/or alert may also provide information about where the subject is at risk of developing the pressure injury, when the subject is at risk of developing the pressure injury, how to adjust a positioning of the subject to prevent the pressure injury, and/or to adjust the positioning of the subject to prevent the fall. In some embodiments, the cloud actionable insight system 185 may notify and/or alert 193 the healthcare professional, caregivers, and/or subject that an area of the subject that is to avoid pressure is currently experiencing pressure and that an adjustment of the positioning of the subject is needed immediately. In some embodiments, the cloud actionable insight system 185 may present 195 to the healthcare professional and/or caregivers via the local computer 130, the user device 160, and/or the management device 170 an individual subject's relevant information. The relevant information may include a visual representation of the subject currently being support by a weight support device 110 and any corresponding pressure injury and/or fall outcomes for that subject. In some embodiments, the relevant information may provide a record of the subject's skin condition throughout their hospital stay for more targeted and efficient care by giving the healthcare providers/caregivers historical subject data that can be used by the cloud actionable insight system 185 for recommendations for treatment.

The continuity of care system environment 105 can translate to better subject outcomes due to the visibility of the subject's condition across every stage of their clinical journey. In an example, the cloud actionable insight system 185 can track the subject at arrival (e.g., on a first mattress), at pre-operation (e.g., on a second mattress), during surgery (e.g., on a third mattress), during post-op (e.g., on a fourth mattress), and at home (e.g., with a take-home mattress). In this example, the cloud actionable insight system 185 (e.g., via the pressure injury outcome engine 150) may determine the subject became at risk of developing a pressure injury on their right hip during surgery, as such the cloud actionable insight system 185 notified/alerted 193 the nurse in post-op to position the subject such that the right hip of the subject will have little to no pressure. In post-op, the cloud actionable insight system 185 determines the subject is at risk of developing a pressure injury on their right shin, as such the cloud actionable insight system 185 notified/alerted 193 the nurse in post-op to adjust the positioning of the subject such that the right shin experiences little to no pressure. At home, the cloud actionable insight system 185 recommends to the subject or other caregiver to position the subject on their left side to allow the subject's body parts that were at risk of developing pressure injury to recover. In this example, the cloud actionable insight system 185 (e.g., via the fall outcome engine 152) may determine the subject in post-op is at risk of falling off of the weight support device 110 via the subject's left side on several occasions (e.g., at three different time instances), as such the cloud actionable insight system 185 notified/alerted 193 the nurse in post-op to adjust the positioning of the subject so that a fall could be avoided. At home, the cloud actionable insight system 185 recommends to the subject or other caregiver to position the subject with a bolster along their left side to aid in the prevention of the subject becoming at risk of falling or experiencing a fall.

In some embodiments, the cloud actionable insight system 185 may compile one or more compliance reports related to pressure injury outcomes and/or fall outcomes of subject(s). For example, a compliance report may include how many (or a percentage of) subjects that had their positioning adjusted within a specified amount of time after the cloud actionable insight system 185 provided a corresponding notification to a healthcare professional, how many (or a percentage of) subject positioning adjustments that took place without the subject(s) developing any pressure injury, how many (or a percentage of) subject positioning adjustments that took place without the subject(s) experiencing a fall, how many (or a percentage of) subject positioning adjustments that took place where the subject(s) did develop a pressure injury and where the pressure injury developed, how many (or a percentage of) subject positioning adjustments that were initiated by the subject, the healthcare professional, and/or the weight support device. The compliance reports may be provided by the cloud actionable insight system 185 to the cloud data store 179 and/or the on-premise data store 159.

While a hospital and home setting are provided as an example, continuity of care may also be applied in other settings in various embodiments.

### Example Weight Support Device Configurations

FIGS. 2A, 2B, and 2C are block diagrams illustrating cross-sectional views of various example configurations of weight support devices, in accordance with some embodiments. The weight support devices 210, 220, and 230 are examples of the weight support device 110 shown in FIG. 1A. The configurations and layers shown in FIG. 2A through FIG. 2C are for example only. In various embodiments, a weight support device may include different, fewer, or additional layers. Certain orders of the layers may also be changed. Furthermore, the layer configuration in one example may be combined with that in another example. Depending on embodiments, the weight support device may be one-sided or two-sided. One or more layers may be removable from the weight support device for washing and cleaning. Also, while many example embodiments discussed herein have air-permeable structure or features, an embodiment of the weight support device 110 may also be not air-permeable or some of the air-permeable structure may not be present.

Referring to FIG. 2A, the weight support device 210 includes microclimate fabric layers 212 as outer layers, a sensor grid layer 214 as a middle layer, and one or more comfort layers 216 that may be used to sandwich or cover the sensor grid layer 214. The entire weight support device 210 may be wrapped by one or more microclimate fabric layers 212.

A microclimate fabric layer 212 may provide regulation to microclimates such as moisture, air, heat, cooling, humidity to the weight support device 210, particularly for the microclimate that may be formed between its surface and the skin of a person. For example, the microclimate fabric layer 212 may be used to reduce the skin temperature and limit the skin's moisture level. The microclimate fabric layer 212 may be a layer that is intended to be directly in contact with the person. The microclimate fabric layer 212 may be medical-graded. The fabric used may be air permeable and washable. The fabric may be formed from suitable materials such as polyester, polyamide, or a composite fabric. To further reduce build-up of moisture, the fabric may be coated with a water-resistant material.

The comfort layer 216 may be formed from foam or other suitable materials that may be used in mattresses, cushions, or seats. The comfort layer 216 may be located below the microclimate fabric layer 212 and above the sensor grid layer 214 to serve as a cushion layer to reduce the potential discomfort brought by the sensor grid layer 214 that might include more rigid components. The material and the thickness of the comfort layer 216 may be selected based on the sensitivity of the sensor grid layer 214. Although the comfort layer 216 may reduce the sensitivity of the sensor grid layer 214, the material of the comfort layer 216 selected should allow the sensor grid layer 214 to perform measurements such as pressure that are related to the person supported by the weight support device 210. Also, in some embodiments, to make the entire weight support device 210 air permeable, air-permeable material such as foam may be selected. The thickness of the comfort layer 216 may also be adjusted based on a balance of the comfort provided by the comfort layer 216 and the sensitivity of the sensor grid layer 214.

The sensor grid layer 214 includes one or more sensor grids, which may include a plurality of sensing points distributed in a target area or substantially the entire surface of the weight support device 210 for taking measurements at different locations. The sensor grid layer 214 may include a pressure sensor grid that includes an array of pressure sensing elements. The pressure sensing elements may take the form of resistive pressure sensors, fiber-optic pressure sensors, or capacitive pressure sensors. In some embodiments, the sensor grid layer 214 may have a configuration that makes the sensor grid layer 214 air permeable.

In one example embodiment, the sensor grid layer 214 may take the form of a thin and flexible capacitive pressure sensor that includes two types of electrodes: columns and rows. Sinusoidal electrical signals are injected at the column electrodes while attenuated sinusoidal signals are detected at the row electrodes, or vice versa. The layer of column electrodes and the layer of row electrodes may be separated by a compressible dielectric material. As a result of the material being compressed by the weight of the person supported by the weight support system 210, the injected electoral signal is attenuated as the signal passes through the dielectric. The attenuation is measured by sensor electronics. A plurality of sensing points that may be formed at the intersections of the column and row electrodes. By arranging the sensing points on a sensor mat, a matrix of pressure values can be captured.

Referring to FIG. 2B, a second example of a weight support device 220 is illustrated. The weight support device 220 may include additional layers (e.g., a moisture sensor grid layer 228 positioned above the microclimate fabric layer 212) and/or additional sensors compared to the weight support device 210.

The moisture sensor grid layer 228 measures surface moisture data. The moisture sensor grid layer 228 may include a surface moisture sensor grid that includes an array of surface moisture sensing elements. The surface moisture sensing elements may take the form of capacitive surface moisture sensors. The capacitive surface moisture sensors may include two types of electrodes: columns and rows. Sinusoidal electrical signals are injected at the column electrodes while attenuated sinusoidal signals are detected at the row electrodes, or vice versa. The column electrodes and the row electrodes may be separated by a permeable dielectric material. As a result of the material being exposed to moisture, the injected electoral signal is attenuated as the signal passes through the dielectric. The attenuation is measured by sensor electronics. A plurality of sensing points that may be formed at the intersections of the column and row electrodes. By arranging the sensing points on a sensor mat, a matrix of surface moisture values can be captured.

The weight support device 220 may include additional sensors, for example one or more accelerometers 222, one or more thermistors 224, a piezoelectric sensor 226, and one or more inertial measurement unit (IMU) sensors (not shown). The accelerometers 222 and the thermistors 224 may be located above the comfort layer 216 so that the sensors are closer to the person. The accelerometers 222 may be used to monitor the movement of the person. The movement data generated by the accelerometers 222 may be used to deduce certain vital measurements such as the heart rate and the respiration rate. The movement data may also be used to deduce the person's condition, such as the person's sleep condition. The thermistors 224 may be used to directly measure the person's skin temperature or serve as proxies to measure the person's temperature (e.g., by measuring a surface temperature). The piezoelectric sensor 226 may also take the form of a sensor grid and may be used as another pressure sensor that is specialized in making certain measurements. For example, the piezoelectric sensor 226 may be specialized in generating pressure data that can be used to deduce the person's heart rate. On the other hand, the sensor grid layer 214 may generate sensor data that is used to deduce other biometrics, such as respiration rates, and poses. The IMU sensors may be used to directly measure surface contours on the surface of the weight support device 110. For example, a change in the IMU sensor's orientation may directly map to a change in the surface of the weight support device 110. A location with a significant change in surface contour (e.g., a location with a deep valley or tall peak) corresponds to a location with high shear.

The signals (sensor data) from the additional layers and/or sensors shown in the weight support device 220 can supplement the information provided by the matrix of pressure values generated by the sensor grid layer 214 to provide richer information for use in the pressure injury outcome detection system and or fall outcome detection system. Alternatively, or additionally, moisture readings, such as skin moisture levels, may also be measured by a hand-held sensor 115 such as a tissue edema sensor.

Conventional methods for measuring surface moisture include one of the following form factors: (1) a one-time use disposable sticker that changes color when exposed to a certain level of moisture, (2) a reusable rigid prong(s) that measure the change in conductivity of the material they are embedded in that results from changing levels of moisture, and (3) a water detection cable or probe connected to a unit for leak detection. These conventional methods are not ideal as they either involve human intervention for the measurement to take place or are not comfortable for a subject. The moisture sensor grid layer 228 with array of surface moisture sensing elements disclosed herein can provide a multiple, thin, flexible, and reusable form factor with little to no human intervention required for measurements to take place. The form factor may be a single strip/sensing element or a high-resolution matrix of sensing elements.

FIG. 2D is a conceptual diagram that shows several example top views of various weight support devices as bedding systems to illustrate example positions of various sensors locations, in accordance with various embodiments. The sensor grid layer 214 may be located throughout substantially the entire surface of the bedding system. The piezoelectric sensor 226 may be specialized in measuring the heart rates of the person and may be located in the area that corresponds to the person's main body, such as the chest area. For example, in one embodiment, the piezoelectric sensor 226 may be located in the area, lengthwise, between 20% of the length and the midpoint of the length. The accelerometers 222 may be located at the corners of the bedding system to monitor the movement of the person. The thermistors 224 may be distributed throughout the area of the bedding system to measure a surface temperature at different parts of the weight support system. The distribution may be uniform or may be more concentrated in particular target areas. The positions of the sensors may change based on the type of weight support devices. For example, in a seating system, the piezoelectric sensor 226 may be located at the back support of the seating system.

The structure of the weight support device can be changed to meet the implementation needs. For example, for short term monitoring applications, the structure of the weight support device can be simplified to just the microclimate fabric layer 212 and the sensor grid layer 214, as illustrated in FIG. 2C as the weight support device 230. The simplification of the weight support device 230 may reduce the cost of manufacturing and also reduce the thickness of the system for easier storage. For a longer-term but basic monitoring applications, such as in household situations where end users would like to monitor their day-to-day sleep conditions, body position, joint locations, movement monitoring, respiration rate, and heart rate the sensor grid layer 214 may be used (or, in some situations, with the addition of the piezoelectric sensor 226). The simpler weight support device 210 may be used in these situations. For more complex and long-term monitoring applications, such as in intensive care situations or in other hospital settings, where various biometrics may need to be monitored, the addition of the second sensor grid layer 214 and supplementary sensors such as accelerometers 222, thermistors 224, and hand-held sensors 115 may be used to supplement the weight support device 220.

### Individually Adjustable Inflatable Zones

FIG. 3A is a view of an example weight support system 300 including a weight support device 110, in accordance with some embodiments. The weight support device 110 may include an adjustable surface. The weight support device 110 may receive instructions from the local computer 130, the computing server 140, or the management device 170 to adjust the surface such that a positioning of the person supported by the weight support device 110 is adjusted. To allow adjustment of the surface, the weight support device 110 includes multiple inflatable zones (e.g., inflation bladders) that may be inflated or deflated. Each inflatable zone is capable of containing pressurized air. When pressurized, the inflatable zone may support a load in contact with the surface of the inflatable zone. Example configurations and arrangement of inflatable zones are discussed in FIGS. 3B and 3C.

The weight support system 300 includes one or more air pumps 305 configured to provide airflow to one or more inflatable zones. In some embodiments, the air pumps 305 are enclosed in a housing to reduce noise caused by operation the air pump 305. In some embodiments, one air pump 305 is coupled to provide airflow to all inflatable zones of the weight support device. Similarly, multiple air pumps 305 may be included such that each subset of inflatable zones is coupled to a dedicated air pump 305. As such, the operation of an air pump 305 may adjust a subset of inflatable zones independently. In yet another embodiment, each inflatable zone of the weight support device 110 may be coupled to a dedicated air pump 305. Examples of air pump 305 configurations include positive displacement pumps, blowers, and axial fan pumps.

The weight support device may include one or more inlets 309 configured to couple to the air pump 305. The inlet 309 may also be coupled to one or more inflatable zones such that the air pump 305 can cause adjustments (e.g., inflating or deflating) to the inflatable zone. In some embodiments, the inlet 309 is configured as a quick-disconnect coupling. Alternatively, the inlet may connect to one or more air pumps 305 using, for example, a threaded connection or push-lock connection.

The weight support system 300 includes a sensor grid layer 214. In some embodiments, the sensor grid layer 214 is coupled to the surface of the weight support device 110. The sensor grid layer 214 includes a plurality of sensors, as described above in regard to FIGS. 2A-D. While the sensor grid layer 214 is described and depicted as a layer, the sensor grid layer 214 may include sensors located at various locations in a stack of layers comprising the weight support device 110. For example, the sensor grid layer 214 may include a portion of sensors coupled to the surface of the weight support device 110 and another portion of sensors embedded into other layers of the weight support device 110. The sensor grid layer 214 is communicatively coupled to the computer 130, such that location-specific pressure data captured by the sensor grid layer 214 may be received by the computer 130.

The computer 130 is configured to control adjustments of the inflatable zones. The computer 130 may control one or more air pumps 305 to regulate and adjust the inflatable zones. In some embodiments, regulation and adjustments of the inflatable zones are performed by the computer 130 based on location-specific pressure data captured by the sensor grid layer 214. In some embodiments, the computer is communicatively coupled to a transceiver 307 integrated with the weight support device 110. The transceiver 307 may be configured to send and receive data to the computer 130. As such, the computer 130 may be in wireless communication with the weight support device 110 (e.g., using a cloud service or a hospital control center) using the transceiver 307. In some embodiments, the computer may communicate with the weight support device 110 through a wired connection (e.g., the computer may be a bedside subject monitor device in a hospital setting). In another example, the computer 130 may be physically integrated into the weight support device 110 and is configured as an onboard computing system.

FIG. 3B is a top view of an example weight support device 110 comprising subsets of inflatable zones, in accordance with some embodiments. The weight support device 110 may include multiple inflatable zones arranged to support various portions of a subject's body. The configuration and arrangement of inflatable zones may be tailored to subjects having a variety of body types and postures. The arrangement of inflatable zones illustrated in FIG. 3B includes a first subset 340 of longitudinally arranged inflatable zones, a second subset 320 of latitudinally arranged inflatable zones, and a third subset 350 of latitudinally inflatable zones. In some embodiments, inflatable zones of each subset of inflatable zones are arranged in groups (e.g., group A or group 1). The weight support system 300 may be configured to adjust inflatable zones in groups. For example, the computer 130 of the weight support system 300 may determine that group A of the second subset 320A of latitudinally arranged inflatable zones should be inflated, using the air pump 305.

In some embodiments, subsets of inflatable zones of the weight support device 110 are configured to support a specific region of the subject. The subset of inflatable zones may vary in volume or construction (e.g., inflatable zones may be configured for different pressure operating ranges) to facilitate support for different regions of the subject's body. For example, as illustrated in FIG. 3B, the weight support device 110 may be configured to support a subject lying down (e.g., in a horizontal resting position). In this example, the first subset 340 of longitudinally arranged inflatable zones is configured to support a sacral region of a subject. The configuration and grouping of longitudinal inflatable zones 340A, 340B, and 340C may, for example, provide more precise support to portions of the sacral region of the subject. By using three groups instead of two groups, the weight support system 300 may support portions of the sacral region of the subject with improved precision. The second subset 320 and third subset 350 of latitudinally arranged inflatable zones may be configured to support the upper body region and lower body region of a subject, respectively. The configuration and grouping of latitudinally inflatable zones 320A, 320B, 350A, and 350B may, for example, provide adequate support to the respective regions of the subject while reducing complexity associated with a greater number of groups (e.g., three groups) for a subset of inflatable zones.

In some embodiments, the weight support device 110 includes one or more bolster inflatable zones 360. The bolster inflatable zone 360 may provide lateral support to the subject (e.g., preventing a subject from rolling off of the weight support device). In some embodiments, the bolster inflatable zone 360 is adjustable such that the weight support system 300 may inflate or deflate bolster inflatable zones 360 of the weight support device 110. For example, the weight support system 300 may adjust (e.g., deflate) one or more bolster inflatable zones 360 to aid in the ingress or egress of the weight support device 110 for a subject.

In some embodiments, the weight support device 110 may implement alternative group-based support within the upper and lower body subsets depicted in FIG. 3B. The computer 130 may command the air pump 305 and the airflow valves to increase internal pressure in a neighboring group within the second subset 320 when a highest-pressure location corresponds to a group within the second subset 320, for example increasing pressure in group B within 320B when a highest-pressure location corresponds to group A within 320A, to reduce pressure at the highest-pressure location without reducing pressure within the group directly beneath the highest-pressure location. The arrangement in FIG. 3B may also allow a complementary pattern within the third subset 350, for example increasing pressure in group D within 350B when a highest-pressure location corresponds to group C within 350A. Transducers may provide airflow or pressure feedback for each addressed group to the computer 130 to confirm attainment of target zone pressures for the alternating pattern.

In some embodiments, the weight support device 110 may execute independent cycle timers for the second subset 320 and the third subset 350 while a separate sequence addresses the sacral subset 340 shown in FIG. 3B. The computer 130 may alternate support among the longitudinal groups 340A, 340B, and 340C by deflating a selected group to a near-zero pressure or by elevating pressure in adjacent groups to offload a targeted region, with the bolster inflatable zones 360 maintained according to a lateral support setting. Cycle durations, target pressures, and group order may be configured per subset to provide region-specific operation, and cycle execution for the second subset 320 and the third subset 350 may proceed without modification to sacral offloading or heel floatation sequences.

FIG. 3C is a section side view of an example weight support device comprising subsets of inflatable zones, in accordance with some embodiments.

FIG. 3D is a section side view the subsets of inflatable zones, in accordance with some embodiments. The example weight support device 110 illustrated in FIG. 3D contains a similar configuration and arrangement of inflatable zones as the weight support device 110 of FIG. 3B. The weight support device includes a first subset 340 of longitudinally arranged inflatable zones, a second subset 320 of latitudinally arranged inflatable zones, and a third subset 350 of latitudinally inflatable zones. In some embodiments, the weight support device 110 may include one or more bolster inflatable zones 360 (not pictured in FIG. 3D).

In some embodiments, the weight support device 110 includes an adjustable base inflatable zone 390. The adjustable base inflatable zone 390 may be configured to support the plurality of inflatable zones and to adjust the vertical height of the weight support device 110 based on the inflation of the base inflatable zone 390. In some embodiments, each inflatable zone may be coupled to a base inflatable zone 390. As such, the weight support device 110 may include multiple base inflatable zones 390. Multiple base inflatable zones 390 may be adjusted individually, in groups, or collectively by the weight support system 300. The weight support system 300 may collectively adjust one or more base inflatable zones 390 to adjust the vertical height of the weight support device 110. For example, the weight support system 300 may adjust (e.g., deflate) one or more base inflatable zones 390 to aid in the ingress or egress of the weight support device 110 for a subject.

In some embodiments, the weight support device 110 may include a layered construction as illustrated in FIG. 3C. A cleanable vapor-permeable cover fabric may overlie a spacer fabric. A microclimate fabric layer 212 may be positioned beneath the spacer fabric. A sensor grid layer 214 may be positioned beneath the microclimate fabric layer 212 and may include sensing elements and associated cabling routed toward an edge bundle. A comfort layer 216 may be positioned beneath the sensor grid layer 214. A support layer comprising mattress air cells and a bottom cover may be positioned beneath the comfort layer 216. One or more hoses coupled to an inlet 309 may route to an air pump 305 to adjust internal pressures of the mattress air cells, and additional hoses for microclimate airflow may be bundled with the inlet 309 as shown in FIG. 3C.

In some embodiments, the mattress air cells of FIG. 3C may correspond to subsets of inflatable zones described with reference numerals 320, 340, and 350. The first subset 340 of longitudinally arranged inflatable zones may be located in a central region to support a sacral region, and the second subset 320 and the third subset 350 of latitudinally arranged inflatable zones may be located toward upper and lower regions. Optional bolster inflatable zones 360 may be positioned laterally, and an optional adjustable base inflatable zone 390 may underlie the subsets of inflatable zones. Connections to airflow valves 410 and transducers 420 may be provided within the support layer scope to enable computer 130 control of group-level and zone-level adjustments through the air pump 305 as depicted in FIG. 3C.

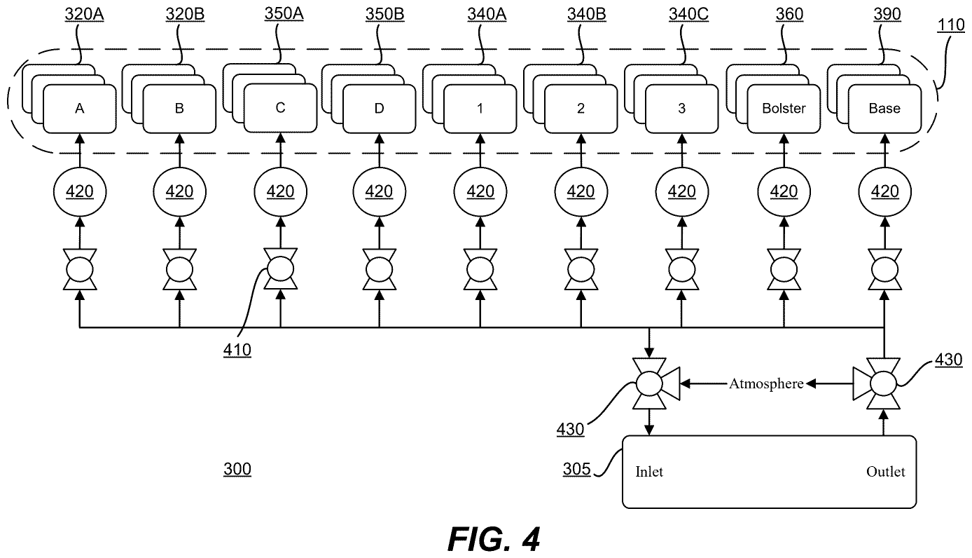

FIG. 4 is a block diagram illustrating an example weight support system 300 including a weight support device 110, in accordance with some embodiments. The weight support system 300 includes a weight support device 110 containing multiple inflatable zones. The example weight support device 110 illustrated in FIG. 4 contains a similar configuration and arrangement of inflatable zones as the weight support device 110 of FIGS. 3B and 3C. As such, the weight support device 110 includes subsets of inflatable zones configured to support specific regions of a subject's body. These subsets are further grouped into groups (e.g., Group A or Group B) to facilitate greater precision in supporting respective portions of the subject's body. For example, as illustrated in FIG. 4, group A 320A and group B 320B are configured to support an upper body region of the subject and group C 350A and group D 350B are configured to support a lower body region of the subject. Similarly, group 1 340A, group 2 340B, and group 3 340C are configured to support a sacral region of the subject. The multiple bolster inflatable zones 360 and base inflatable zones 390 are configured to provide vertical support for inflatable zones of the weight support device 110 surface and lateral support for the subject, respectively.

In some embodiments, the weight support system 300 includes multiple airflow valves 410 and multi-port airflow valves 430. The weight support system 300 may use the airflow valves 410 to adjust inflatable zones of the weight support device. An airflow valve 410 may, for example, restrict, meter, or allow the passage of airflow to and from an inflatable zone. For example, the airflow valve 410 may restrict airflow delivered from the air pump 305 to one inflatable zone, thereby diverting the airflow to other inflatable zones of the weight support device 110. In some embodiments, each group of inflatable zones of a subset of inflatable zones is coupled to an associated air valve 410. As such, the weight support system 300 can adjust all inflatable zones of a group by controlling the associated air valve 410. For example, the weight support system 300 might lower the vertical height of the weight device 110 by adjusting all base inflatable zones 390 using an airflow valve 410 associated with the base group. In embodiments including multi-port airflow valves 430, airflow may flow to and from the atmosphere. For example, a multi-port airflow valve 430 may include a port that vents to atmosphere. As such, an air pump 305 may intake air from the atmosphere or vent air from the weight support system 300 using a multi-port airflow valve 430. For example, the weight support system 300 may deflate one or more inflatable zones by venting airflow from an adjustable zone to the atmosphere.

In some embodiments, the weight support system 300 includes one or more transducers 420. The weight support system 300 may use one or more transducers 420 to capture data (e.g., pressure or flow rate data) describing airflow within the weight support system 300. Airflow data may be received by the computer 130 to monitor adjustments of inflatable zones of the weight support device. In some embodiments, each group of inflatable zones of a subset of inflatable zones is coupled to an associated transducer 420. In another example, a subset of inflatable zones is coupled to an associated transducer 420 such that all groups of the subset are monitored by one transducer 420.

### Example Zone Specific Pressure Relief Process

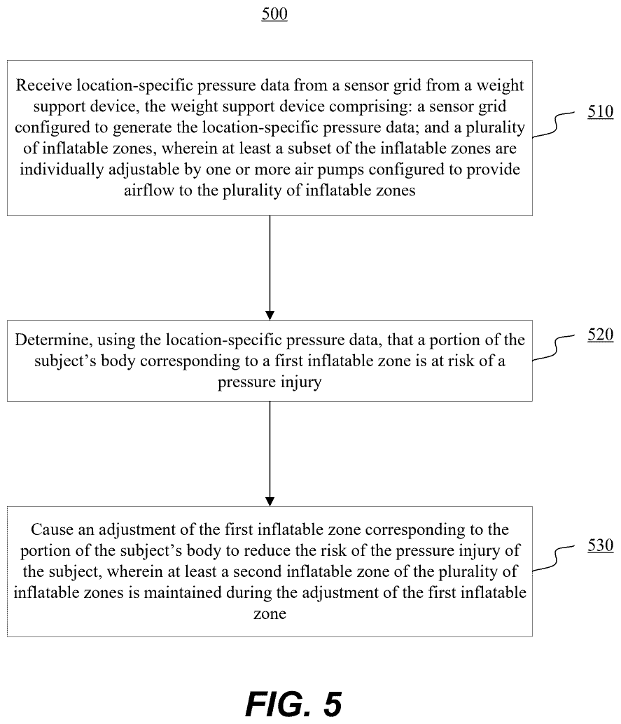

FIG. 5 is a flowchart depicting an example process 500 for performing a zone specific pressure relief process for a subject supported by the weight support device 110, in accordance with some embodiments. A computer, which may be the computing server 140, the local computer 130, or the user device 160, may perform the process 500. Other entities may perform some or all of the steps in FIG. 5 in other embodiments. Embodiments may include different and/or additional steps or perform the steps in different orders.

The weight support system 300 receives 510 location-specific pressure data from the weight support device 110. For example, the weight support system 300 may receive the pressure data from the sensor grid layer 214 of the weight support device 110. The pressure data may be raw pressure data and include a time series of matrix readings. The pressure data may also be processed data whose time series has been digitally filtered by various digital signal processing techniques such as a finite impulse response (FIR) filter, Gaussian filter, smoothing, etc. For each time instance, the pressure data may include two-dimensional data that corresponds to the sensor grid layer 214 of the weight support device 110. In some embodiments, the weight support system 300 may also receive additional data such as surface moisture data, individual's vital data, temperature data, and other suitable data.

The weight support system 300 determines 520, using the location-specific pressure data, that a portion of the subject's body corresponding to a first inflatable zone is at risk of a pressure injury. In some embodiments, the weight support system 300 may process the location-specific pressure data from the sensor grid layer 214 to derive a contact area, a body outline, position classifications, and peak pressure metrics using the sensor mapping engine 142 and the machine vision engine 144. The computer 130 may apply a rules-based threshold and/or a machine learning model executed by the machine learning engine 146 and the pressure injury outcome engine 150, optionally using surface moisture data, surface temperature data, and health record data from the data store 155, to determine that the portion of the subject's body mapped to a first inflatable zone exceeds a risk threshold for pressure injury.

In some embodiments, the weight support system 300 may associate the mapped portion with a group within the first subset 340, the second subset 320, or the third subset 350 shown in FIG. 3B and may record a risk score, a timestamp, and an identifier for the first inflatable zone in the data store 155. The computer 130 may prepare targets for the air pump 305 and the airflow valves 410 and may select monitoring channels from the transducers 420 for a subsequent adjustment process 530 that maintains inflation of at least a second inflatable zone while addressing the first inflatable zone.

The weight support system 300 causes 530, an adjustment of the inflatable zone corresponding to the portion of the subject's body to reduce the risk of the pressure injury of the subject. To maintain support of the subject, at least a second inflatable zone maintains inflation during the adjustment of the inflatable zone. In some embodiments, the second inflatable zone is adjacent to the inflatable zone such that the first inflatable zone and the second inflatable zone are arranged in an alternating pattern.

### Example Pressure Injury Outcome Detection Processes

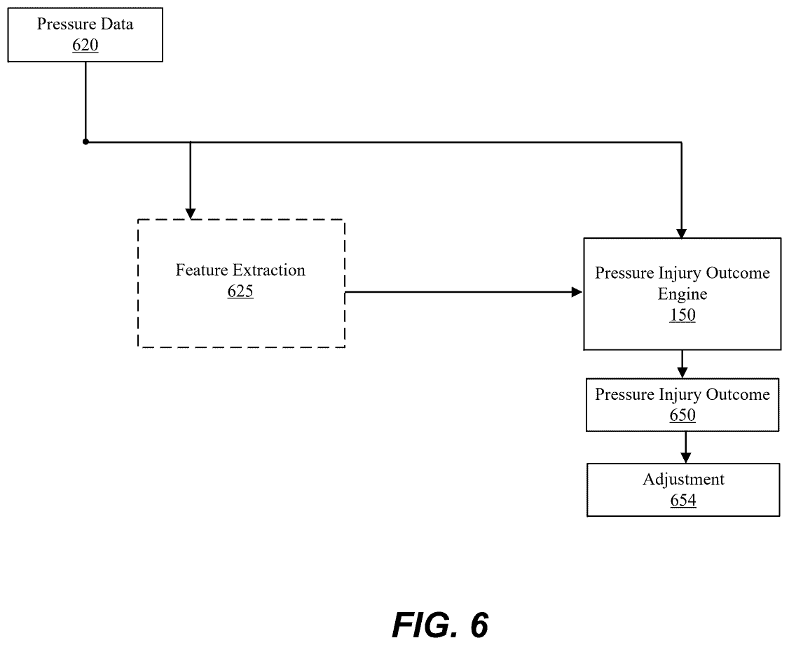

FIG. 6 is a block diagram illustrating an example algorithmic pipeline for predicting a pressure injury outcome 650 for a person, in accordance with some embodiments. The pipeline includes an input (e.g., pressure data 620), a feature extraction stage 625, and outputs of the pipeline.

The input may include pressure data 620. The pressure data 620 includes raw sensor readings currently being collected by a weight support device (e.g., the weight support device 110) as described above.

In some embodiments, the input is provided directly to the pressure injury outcome engine 150. In some embodiments, the input is provided to the feature extraction stage 625. The feature extraction stage 625 may perform operations discussed above in reference to the sensor mapping engine 142, the machine vision engine 144, the machine learning engine 146, and the vital analysis engine 148. The feature extraction stage 625 extracts particular features which may be input into the pressure injury outcome engine 150. The extracted features may include various position data 660 associated with the person (e.g., a pose of the person, a position of the person, joint locations of the person, and movement data), pressure data at particular body part locations with respect to the person, determined one or more areas of high shear at particular body part locations with respect to the person and risk factors of the person that may contribute to developing a pressure injury.

The feature extraction stage 625 may analyze the input to determine one or more risk factors of the person that may contribute the person developing a pressure injury. Some example methods for determining risk factors are discussed in U.S. Pat. No. 9,320,665, patented on Apr. 26, 2016, entitled “Risk Modeling for Pressure Ulcer Formation,” which is incorporated by reference herein for all purposes.

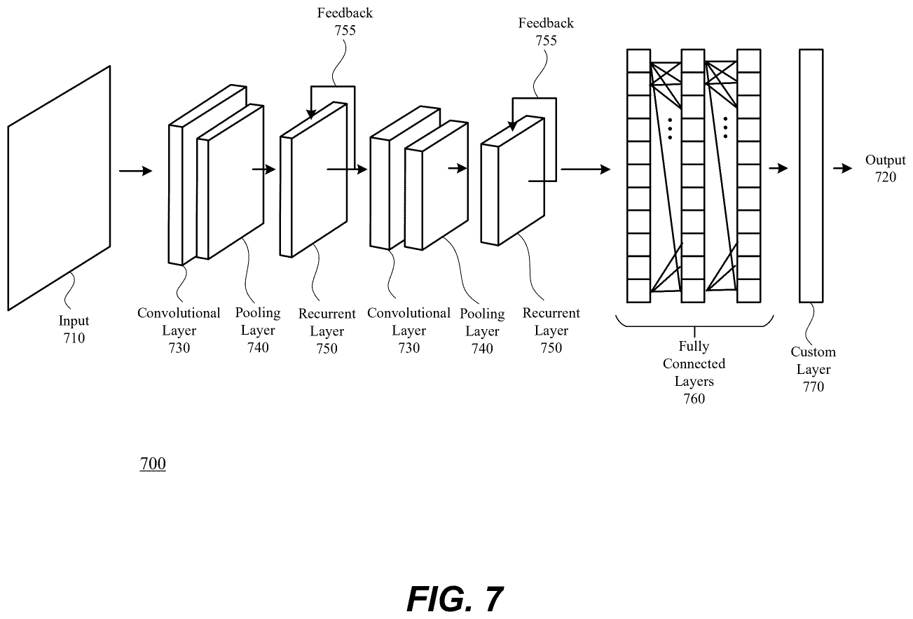

The feature extraction stage 625 may analyze the input to determine various pressure injury outcomes 650 associated with the person by utilizing machine learning models discussed in detail with reference to FIG. 7.

The feature extraction stage 625 may analyze the input to determine areas of overlap. For example, the feature extraction stage 625 may utilize a body outline and/or the model of the person as discussed above and analyze the pressure data 620 to find particular locations where the person's body outline and/or model overlaps with areas of high pressure in the determination of the pressure injury outcome 650 of the person. As such, pressure data at particular body part locations with respect to the person at particular body part locations with respect to the person. In some embodiments, the feature extraction stage 625 may weight areas of overlap higher based on one or more timing parameters. The timing parameters may include how long the areas have overlapped and/or how long the areas have overlapped over a specified time interval.

The pressure injury outcome engine 150 determines the pressure injury outcome 650 for the person. In some embodiments, the pressure injury outcome engine 150 utilizes a rules-based approach and/or a machine learning model (e.g., a CNN) to determine the pressure injury outcome 650. For example, the input may be directly fed into the machine learning model. In another example, the extracted features from the feature extraction stage 625 are fed into the machine learning model. In some embodiments, the machine learning model may be trained on a combination of historical input data and known pressure injury outcomes (e.g., historical subject data).

The pressure injury outcome 650 as described above may include a risk of the person developing a pressure injury, an area of the person's body at risk of developing the pressure injury, and an amount of time that indicates when an adjustment 654 of a positioning of the person is needed to avoid pressure injury.

### Targeted Sacral Offloading

In some embodiments, the weight support device 110 generates sensor signals communicated with a computer 130 to automatically detect a pressure injury outcome of a subject. The weight support device 110 may measure the pressure exerted by the subject using the sensor grid layer 214 to generate a matrix of pressure readings. The matrix of pressure readings and other sensor readings (e.g., surface moisture readings), which may be supplemented with other supporting sensors, may be provided to a computer with an artificial intelligence system which uses one or more types of machine learning networks (a convolutional neural network (CNN), a long short term memory (LSTM) network, etc.) to identify the pressure injury outcome (e.g., a risk of the person developing a pressure injury). The matrix of pressure readings and other sensor readings may also be used to deduce other information about the person that may include, but is not limited to, respiration rate, heart rate, and/or position data that includes body position, joint locations, and movement monitoring.

In some embodiments, the weight support system 300 is configured to reduce the risk of a pressure injury identified for a subject using the weight support device 110. With the subject supported by the weight support device 110, the computer 130 detects the presence of the subject based on pressure data from the sensor grid layer 214. The weight support system 300 may initially maintain a preset internal air pressure for the inflatable zones (e.g., for a period of time following detecting the subject). For example, the air pressure for the inflatable zones may be configured for an optimum redistribution of pressure on the body to support all regions of the subject's body. Similarly, the preset internal air pressure may be set based on a desirable degree of comfort for the subject. In some embodiments, the weight support device 110 maintains preset internal zone pressures until the subject is detected, using the sensor grid layer 214, by the weight support system 300.

Following the period of time at the preset air pressure for the inflatable zones, the weight support system 300 may continuously monitor sensor grid layer 214 readings from the surface of the weight support device. The risk of the subject developing a pressure injury may be determined, for example, using a threshold (e.g., defining an elevated pressure event as a pressure reading above 32 mmHg) or using a machine-learning model trained to classify body parts at risk of developing a pressure injury. The configuration of such a machine-learning model is described in regard to FIG. 7. The elevated pressure event corresponds to a location on the sensor grid layer 214. Depending on the arrangement of inflatable zones in the weight support device 110, the location of the elevated pressure event on the sensor grid layer 214 may correspond to one or more inflatable zones.

If the location of the elevated pressure event corresponds to the sacral region of a subject, the weight support system 300 may adjust inflatable zones to perform sacral offloading for the subject. Sacral offloading involves resolving an elevated pressure event or reducing pressure, using the weight support system 300, for the sacral region of a subject. In the example weight support device 110 illustrated in FIG. 3B, the weight support device 110 includes a subset 340 of longitudinally arranged inflatable zones configured to support the sacral region of a subject. In this example, groups of the longitudinally arranged inflatable zones can be adjusted to reduce pressure on portions of the sacral region of the subject's body and address the elevated pressure event. In some embodiments, the weight support system deflates one group (e.g., group 2) of the longitudinally arranged inflatable zones until the pressure (e.g., measured by the sensor grid layer 214) at the location of the elevated pressure event is approximately zero (e.g., <5 mmHg). This threshold may be adjustable or dynamically set (e.g., using the machine learning model). To maintain support for the subject, one or more groups (e.g., group 1 and group 3) remain inflated at a greater internal zone pressure than the deflated group. This coordinated adjustment of groups of inflatable zones provides sacral offloading of the elevated pressure event location while maintaining support for the subject. In some embodiments, the group of inflatable zones is deflated for a period of time (e.g., 10 minutes) after which the inflatable zones are inflated to the preset internal zone pressures.

In some embodiments, sacral offloading may occur for the highest pressure location on the sensor grid 214 in the sacral region of the subject, regardless of the pressure setpoint. For example, the weight support system 300 may initiate sacral offloading at a preset interval (e.g., every 10 minutes) regardless of whether pressure readings of the sensor grid layer 214 exceed the preset threshold. Sacral offloading may occur for a specific period of time, after which the inflatable zones are inflated to the preset internal zone pressures.

### Targeted Heel Floatation

If the location of an elevated pressure event corresponds to the lower body region (e.g., one or both heels) of a subject, the weight support system 300 may adjust inflatable zones to perform lower body floatation for the subject. Lower body floatation involves elevated pressure event or reducing pressure, using the weight support system 300, for the lower body region of a subject. In the example weight support device 110 illustrated in FIG. 3B, the weight support device 110 includes a subset 350 of latitudinally arranged inflatable zones configured to support the lower body region of a subject. In this example, groups of the latitudinally arranged inflatable zones can be adjusted to change the pressure on portions of the lower body region of the subject's body and address the elevated pressure event. In some embodiments, the weight support system deflates one group (e.g., group C) of the latitudinally arranged inflatable zones until the pressure (e.g., measured by the sensor grid layer 214) at the location of the elevated pressure event is approximately zero (e.g., <5 mmHg). To maintain support for the subject, another group (e.g., group D) remains inflated at a greater internal zone pressure than the deflated group. Similarly, the other group may be inflated at a higher internal zone pressure than the current preset internal zone pressure of the first group to provide additional support to the lower body region (e.g., one or both heels) of the subject. This coordinated adjustment of groups of inflatable zones provides lower body floatation while maintaining support for the subject. In some embodiments, the group of inflatable zones is deflated for a period of time (e.g., 10 minutes) after which the inflatable zones are inflated to the preset internal zone pressures.

### Smart Alternating Pressure Zones

In some embodiments, the weight support system 300 may adjust inflatable zones to reduce pressure for the upper or lower regions of the subject independently from other regions. In the example weight support device 110 illustrated in FIG. 3B, the weight support device 110 includes a subset 320 of latitudinally arranged inflatable zones configured to support the upper body region of the subject and a subset 350 of latitudinally arranged inflatable zones configured to support the lower body region of the subject. In this example, groups of the subsets of latitudinally arranged inflatable zones can be adjusted to reduce pressure on portions of the upper and lower regions of the subject's body. In some embodiments, these groups may be adjusted to address an elevated pressure event. The weight support system may inflate one group (e.g., group B) of the longitudinally arranged inflatable zones until the pressure (e.g., measured by the sensor grid layer 214) at the location of the elevated pressure event (e.g., corresponding to group A) is approximately zero (e.g., <5 mmHg). By increasing pressure in one or more inflatable zone groups adjacent to the elevated pressure event, the pressure at the elevated pressure event location is effectively reduced without deflating the inflatable zone corresponding to that location. This coordinated adjustment of groups of inflatable zones provides floatation that supports the portion of the subject's body corresponding to the elevated pressure event location. In some embodiments, the one or more groups of inflatable zones is inflated for a period of time (e.g., 10 minutes) after which the inflatable zones are inflated to the preset internal zone pressures.

In some embodiments, floatation or offloading may occur for the highest pressure location on the sensor grid 214 in the upper or lower body region of the subject. For example, the weight support system 300 may initiate floatation for one or more portions of the upper or lower body regions at a preset interval (e.g., every 10 minutes) regardless of whether pressure readings of the sensor grid layer 214. In some embodiments, groups of subsets of inflatable zones may alternate support for the subject in an inflation and deflation cycle. For the period of the cycle, groups of inflatable zones may inflate and deflate such that an adjacent group of inflatable zones supports the subject while the other group of inflatable zones is deflated. In some embodiments, the floatation or offloading of the upper and lower body regions occurs independently of other cycles (e.g., sacral offloading or heel floatation, as described above).

### Example Architecture of Machine Learning Models

In various embodiments, a wide variety of machine learning techniques may be used for detection of a pressure injury outcome, a person's pose such as side labels and joint locations, identification of the person's outline, identification of potential seizure, identification of imminent fall, and other uses described herein. The machine learning techniques include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN) and recurrent neural networks (RNN) (e.g., long short-term memory networks (LSTM)), may also be used.

In various embodiments, the training techniques for a machine learning model may be supervised, semi-supervised, or unsupervised. In supervised learning, the machine learning models may be iteratively trained with a set of training samples that are labeled. For example, for a machine learning model trained to classify body parts at risk of developing a pressure injury, the training samples may be different heatmaps of pressure data labeled with body parts that did and did not develop pressure injuries. Thus, the labels for each training sample may be binary or multi-class.

In another example for iteratively training a machine learning model to determine when a person should have their position adjusted to avoid a pressure injury, the training samples may be historical data of individuals who developed a pressure injury (e.g., each individual's pressure data and health record). For training a binary machine learning model (e.g., a model that identifies whether a person develops pressure injury, whether a particular body part of a person develops a pressure injury, etc.), training samples may include a positive training set (with training samples that have the label of having a pressure injury) and a negative training set (with training samples that have the label of not having a pressure injury). In some cases, an unsupervised learning technique may be used. The samples used in training are not labeled. Various unsupervised learning techniques such as clustering may be used. In some cases, the training may be semi-supervised with the training set having a mix of labeled samples and unlabeled samples.

A machine learning model may be associated with an objective function, which generates a metric value that describes the objective goal of the training process. For example, the training may intend to reduce the error rate of the model in generating predictions. In such a case, the objective function may monitor the error rate of the machine learning model. In object recognition (e.g., object detection and classification), the objective function of the machine learning algorithm may be the training error rate in classifying objects in a training set. Such an objective function may be called a loss function. Other forms of objective functions may also be used, particularly for unsupervised learning models whose error rates are not easily determined due to the lack of labels. In pressure injury outcome detection, the objective function may correspond to the difference between the model's prediction of a person developing a pressure injury and the manually identified development of a pressure injury in the training sets. In fall outcome detection, the objective function may correspond to the difference between the model's prediction that a person may experience a fall and the manually identified fall(s) experienced by the person in the training set. In various embodiments, the error rate may be measured as cross-entropy loss, L1 loss (e.g., the sum of absolute differences between the predicted values and the actual value), L2 loss (e.g., the sum of squared distances).

FIG. 7 shows an example structure of a neural network, which may include layers that may present in various machine learning models. For example, a CNN may include the convolutional layers and the pooling layers shown in FIG. 7. An LSTM may include the recurrent layers shown in FIG. 7. Each machine learning models may have its own structure and layers (while omitting some layers in FIG. 7). The order of the layers in FIG. 7 is also an example. The order of layers may change, depending on the type of machine learning model used.

Referring to FIG. 7, a structure of an example neural network (NN) is illustrated, according to an embodiment. The NN 700 may receive an input 710 and generate an output 720. The NN 700 may include different kinds of layers, such as convolutional layers 730, pooling layers 740, recurrent layers 750, full connected layers 760, and custom layers 770. A convolutional layer 730 convolves the input of the layer (e.g., an image) with one or more kernels to generate different types of images that are filtered by the kernels to generate feature maps. Each convolution result may be associated with an activation function. A convolutional layer 730 may be followed by a pooling layer 740 that selects the maximum value (max pooling) or average value (average pooling) from the portion of the input covered by the kernel size. The pooling layer 740 reduces the spatial size of the extracted features. In some embodiments, a pair of convolutional layer 730 and pooling layer 740 may be followed by a recurrent layer 750 that includes one or more feedback loop 755. The feedback 755 may be used to account for spatial relationships of the features in an image or temporal relationships of the objects in the image. The layers 730, 740, and 750 may be followed in multiple fully connected layers 760 that have nodes (represented by squares in FIG. 7) connected to each other. The fully connected layers 760 may be used for classification and object detection. In one embodiment, one or more custom layers 770 may also be presented for the generation of a specific format of output 720. For example, a custom layer may be used for image segmentation for labeling pixels of an image input with different segment labels.

The order of layers and the number of layers of the NN 700 in FIG. 7 is for example only. In various embodiments, a NN 700 includes one or more convolutional layers 730 but may or may not include any pooling layer 740, recurrent layer 750, or fully connected layers 760. If a pooling layer 740 is present, not all convolutional layers 730 are always followed by a pooling layer 740. A recurrent layer may also be positioned differently at other locations of the CNN. For each convolutional layer 730, the sizes of kernels (e.g., 3×3, 5×5, 7×7, etc.) and the numbers of kernels allowed to be learned may be different from other convolutional layers 730.

A machine learning model may include certain layers, nodes, kernels and/or coefficients. Training of a neural network, such as the NN 700, may include forward propagation and backpropagation. Each layer in a neural network may include one or more nodes, which may be fully or partially connected to other nodes in adjacent layers. In forward propagation, the neural network performs the computation in the forward direction based on outputs of a preceding layer. The operation of a node may be defined by one or more functions. The functions that define the operation of a node may include various computation operations such as convolution of data with one or more kernels, pooling, recurrent loop in RNN, various gates in LSTM, etc. The functions may also include an activation function that adjusts the weight of the output of the node. Nodes in different layers may be associated with different functions.

Each of the functions in the neural network may be associated with different coefficients (e.g. weights and kernel coefficients) that are adjustable during training. In addition, some of the nodes in a neural network may also be associated with an activation function that decides the weight of the output of the node in forward propagation. Common activation functions may include step functions, linear functions, sigmoid functions, hyperbolic tangent functions (tanh), and rectified linear unit functions (ReLU). After an input is provided into the neural network and passes through a neural network in the forward direction, the results may be compared to the training labels or other values in the training set to determine the neural network's performance. The process of prediction may be repeated for other images in the training sets to compute the value of the objective function in a particular training round. In turn, the neural network performs backpropagation by using gradient descent such as stochastic gradient descent (SGD) to adjust the coefficients in various functions to improve the value of the objective function.

Multiple rounds of forward propagation and backpropagation may be performed. Training may be completed when the objective function has become sufficiently stable (e.g., the machine learning model has converged) or after a predetermined number of rounds for a particular set of training samples. The trained machine learning model can be used for performing a pressure injury outcome detection, body part detection, joint identifications, fall outcome detection, or another suitable task for which the model is trained.

### Example Region Specific Offload Process

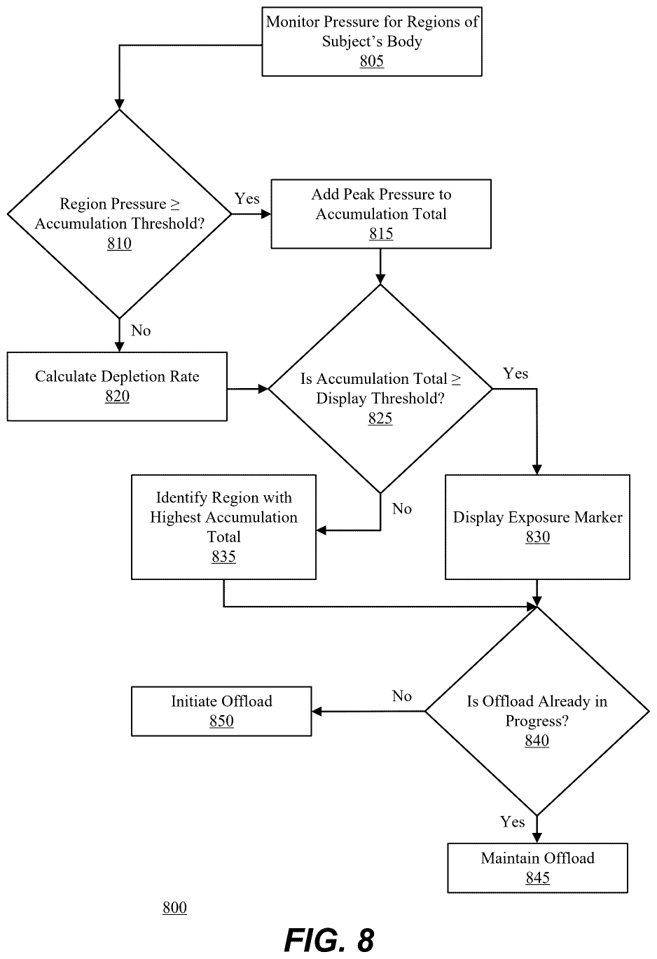

FIG. 8 is a flowchart depicting an example region-specific offload process, in accordance with some embodiments. In some embodiments, the weight support device 110 may execute a region-specific offload process 800. The weight support device 110 may monitor pressures for predefined body regions at step 805 using the sensor grid layer 214 and may generate location-specific pressure data mapped to an upper body region supported by the second subset 320, a sacral region supported by the first subset 340, and a lower body region supported by the third subset 350. The weight support device 110 may generate regional peak pressure values and average peak pressure values over a time window and may maintain per-region accumulation totals that represent exposure over time.

In some embodiments, the weight support device 110 may compare a regional pressure value to an accumulation threshold at step 810. When a regional pressure value meets or exceeds the accumulation threshold, the weight support device 110 may add a peak pressure contribution to a corresponding accumulation total at step 815. When a regional pressure value remains below the accumulation threshold, the weight support device 110 may calculate a depletion rate at step 820 to reduce the accumulation total over time. The weight support device 110 may weight accumulation and depletion using configurable parameters that may be based on body position, surface moisture, or subject-specific settings maintained by the computer 130.

In some embodiments, the weight support device 110 may evaluate whether a per-region accumulation total meets or exceeds a display threshold at step 825. When the display threshold is met, the weight support device 110 may present an exposure marker at step 830 through an interface 165 on a user device 160 or a management device 170. The weight support device 110 may provide the identified region, an exposure score derived from the accumulation total, and a timestamp that associates the exposure marker with the monitoring interval.

In some embodiments, the weight support device 110 may identify at step 835 a region with a highest accumulation total among monitored regions and may query at step 840 whether an offload is already in progress for the identified region. When an offload is already in progress, the weight support device 110 may maintain offload at step 845 by holding target pressures for the previously selected inflatable zones while continuing accumulation tracking for other regions. The weight support device 110 may maintain lateral support through bolster inflatable zones 360 and may maintain vertical height through an adjustable base inflatable zone 390 during the maintenance interval.

In some embodiments, when no offload is in progress for the identified region, the weight support device 110 may initiate offload at step 850 by commanding the air pump 305 and the airflow valves 410 to adjust inflatable zones associated with the region with the highest accumulation total. The weight support device 110 may reduce pressure in a first inflatable zone within group 340B and maintain pressure in adjacent groups 340A and 340C for sacral offloading, may reduce pressure in group 350A while maintaining pressure in group 350B for heel floatation, or may increase pressure in group 320B while maintaining pressure in group 320A to provide floatation for an upper body region. The weight support device 110 may verify commanded pressures using transducers 420 and may maintain inflation in at least a second inflatable zone during each adjustment to preserve regional support. The weight support device 110 may update the accumulation totals and exposure markers after the offload interval and may repeat evaluation from step 805 for continuous operation.

### Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Embodiments according to the invention are in particular disclosed in the attached claims directed to a method and a computer program product, wherein any feature mentioned in one claim category, e.g. method, can be claimed in another claim category, e.g. computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject-matter which can be claimed comprises not only the combinations of features as set out in the disclosed embodiments but also any other combination of features from different embodiments. Various features mentioned in the different embodiments can be combined with explicit mentioning of such combination or arrangement in an example embodiment. Furthermore, any of the embodiments and features described or depicted herein can be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines may be embodied in software, firmware, hardware, or any combinations thereof.

Any of the steps, operations, or processes described herein may be performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure may describe a process that includes multiple steps sequentially with arrows present in a flowchart, the steps in the process do not need to be performed by the specific order claimed or described in the disclosure. Some steps may be performed before others even though the other steps are claimed or described first in this disclosure.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

What is claimed is:

1. A weight support system for a subject, the weight support system comprising:
a weight support device configured to support the subject, the weight support device comprising:
a sensor grid configured to generate location-specific pressure data; and
a plurality of inflatable zones, wherein at least a subset of the inflatable zones are individually adjustable;
one or more air pumps configured to provide airflow to the plurality of inflatable zones; and
a computer configured to control adjustments of the plurality of inflatable zones, wherein controlling the adjustments of the plurality of inflatable zones comprises:
receiving the location-specific pressure data from the sensor grid;
determining, using the location-specific pressure data, that a portion of the subject's body corresponding to a first inflatable zone is at risk of a pressure injury; and
causing an adjustment of the first inflatable zone corresponding to the portion of the subject's body to reduce the risk of the pressure injury of the subject, wherein at least a second inflatable zone of the plurality of inflatable zones is maintained during the adjustment of the first inflatable zone.

2. The weight support system of claim 1, further comprising a plurality of transducers, wherein each transducer is configured to monitor an adjustment of one of the plurality of inflatable zones.

3. The weight support system of claim 1, further comprising a plurality of airflow valves, wherein each airflow valve is configured to control airflow to one of the plurality of inflatable zones.

4. The weight support system of claim 1, wherein the plurality of inflatable zones comprises:
a first subset of longitudinally arranged inflatable zones configured to support a sacral region of the subject;
a second subset of latitudinally arranged inflatable zones configured to support an upper body region of the subject; and
a third subset of latitudinally arranged inflatable zones configured to support a lower body region of the subject.

5. The weight support system of claim 1, wherein the computer is configured to control adjustments for the plurality of inflatable zones using a machine-learning model, wherein the machine-learning model is configured to receive the location-specific pressure data and outputs a prediction describing one or more locations on the sensor grid that exceed a pressure threshold and the portion of the subject's body corresponding to the one or more locations.

6. The weight support system of claim 1, wherein the computer is configured to control adjustments for the plurality of inflatable zones using a machine-learning model, wherein the machine-learning model is trained to identify that a portion of the subject's body is at risk of a pressure injury based on the pressure data for one or more locations on the sensor grid.

7. A method for supporting a subject using a plurality of inflatable zones comprising:
receiving location-specific pressure data from a sensor grid from a weight support device, the weight support device comprising:
a sensor grid configured to generate the location-specific pressure data; and
a plurality of inflatable zones, wherein at least a subset of the inflatable zones are individually adjustable by one or more air pumps configured to provide airflow to the plurality of inflatable zones;
determining, using the location-specific pressure data, that a portion of the subject's body corresponding to a first inflatable zone is at risk of a pressure injury; and
causing an adjustment of the first inflatable zone corresponding to the portion of the subject's body to reduce the risk of the pressure injury of the subject, wherein at least a second inflatable zone of the plurality of inflatable zones is maintained during the adjustment of the first inflatable zone.

8. The method of claim 7, further comprising:
monitoring adjustments to the plurality of inflatable zones using a plurality of transducers.

9. The method of claim 7, wherein causing an adjustment of the first inflatable zone comprises controlling airflow for adjustments to the plurality of inflatable zones using a plurality of airflow valves.

10. The method of claim 7, wherein the subset of the inflatable zones is configured to support at least a sacral region, an upper body region, or a lower body region of the subject.

11. The method of claim 7, wherein determining that a portion of the subject's body is at risk of a pressure injury comprises using a machine-learning model, wherein the machine-learning model is configured to receive location-specific pressure data and outputs a prediction describing one or more locations on the sensor grid that exceed a pressure threshold and the portion of the subject's body corresponding to the one or more locations.

12. The method of claim 7, wherein determining that a portion of the subject's body is at risk of a pressure injury comprises using a machine-learning model, wherein the machine-learning model is trained to identify that a portion of the subject's body is at risk of a pressure injury based on the pressure data for one or more locations on the sensor grid.

13. A mattress for a subject, the mattress comprising:
a sensor grid configured to generate location-specific pressure data;
a plurality of inflatable zones, wherein at least a subset of the inflatable zones are individually adjustable;
a plurality of inlets configured to be coupled to one or more air pumps configured to provide airflow to the plurality of inflatable zones; and
a transceiver configured to be in communication with a computer, the computer configured to control adjustments of the plurality of inflatable zones, wherein controlling the adjustments of the plurality of inflatable zones comprises:
receiving the location-specific pressure data from the sensor grid;
determining, using the location-specific pressure data, that a portion of the subject's body corresponding to a first inflatable zone is at risk of a pressure injury; and
causing an adjustment of the first inflatable zone corresponding to the portion of the subject's body to reduce the risk of the pressure injury of the subject, wherein at least a second inflatable zone of the plurality of inflatable zones is maintained during the adjustment of the first inflatable zone.

14. The mattress of claim 13, wherein the first inflatable zone and the second inflatable zone of the plurality of inflatable zones are arranged in an alternating configuration.

15. The mattress of claim 13, wherein the plurality of inflatable zones comprises:
a first subset of longitudinally arranged inflatable zones configured to support a sacral region of the subject;
a second subset of latitudinally arranged inflatable zones configured to support an upper body region of the subject; and
a third subset of latitudinally arranged inflatable zones configured to support a lower body region of the subject.

16. The mattress of claim 15, wherein only the inflatable zones of the first subset of longitudinally arranged inflatable zones configured to support the sacral region of the subject are individually adjustable.

17. The mattress of claim 13, further comprising an adjustable base inflatable zone configured to support the plurality of inflatable zones and to adjust the vertical height of the mattress based on the inflation of the base inflatable zone.

18. The mattress of claim 17, further comprising a plurality of adjustable base inflatable zones.

19. The mattress of claim 13, further comprising an adjustable bolster inflatable zone configured to provide lateral support for the subject.

20. The mattress of claim 19, wherein the mattress comprises a plurality of adjustable bolster inflatable zones.
